(19) United States
(12) Patent Application Publication
(10) Pub. No.: US 2007/0178087 A1
(43) Pub. Date: Aug. 2, 2007

(54) Method for Treating Immune Dysfunction by Regulation of CD40 Ligand Expression

(76) Inventors: Christopher Roman, New York, NY (US); Chongmin Huan, Brooklyn, NY (US)

Correspondence Address: Ryan, Mason & Lewis, LLP, 90 Forest Avenue, Locust Valley, NY, 11560, US

(21) Appl. No.: 11/610,920

(22) Filed: Dec. 14, 2006

Related U.S. Application Data
(60) Provisional application No. 60/750,611, filed on Dec. 15, 2005.

(51) Int. Cl. A61K 48/00; A61K 39/395

(52) U.S. Cl. 424/143.1; 514/44

(57) ABSTRACT

Techniques for treating immune dysfunction in a patient by regulation of CD40 Ligand (CD40L) expression are provided. For example, one technique includes suppressing TFE3 and/or TFEB in the patient to thereby suppress CD40L expression in the patient. In addition, the technique may include administering a TFE3 inhibitor and/or a TFEB inhibitor for suppressing TFE3 and/or TFEB, respectively, in the patient.

### CROSS-REFERENCE TO RELATED APPLICATION(S)

This application claims the benefit of U.S. Provisional Application Ser. No. 60/750,611, filed on Dec. 15, 2005, the disclosure of which is incorporated by reference herein.

### FIELD OF THE INVENTION

The present invention relates to immunology and, more particularly, to the treatment of immune dysfunction by the regulation of CD40 ligand (CD40L) expression.

### BACKGROUND OF THE INVENTION

The immune system is a responsive protection system comprised of cells in bone marrow, the thymus, and the lymphatic system of ducts and nodes, spleen, and blood. Aspects of the immune system include the innate immune response and the adaptive immune response. An innate immune response is nonspecific, and includes, for example, skin, cilia in mucous membranes, tears, saliva, nasal secretions, and phagocytic cells that migrate to infected areas and engulf pathogens. An adaptive immune response is a specific response against an individual antigen, and includes humoral and cellular systems. The humoral system produces antibodies to eliminate pathogens and their products. The cellular system eliminates pathogens that have invaded cells and regulates the body's entire immune response.

The humoral system response involves B cells, which are specialized white blood cells produced in the bone marrow. Every B cell contains multiple copies of one kind of antibody as a surface receptor for antigen. When the antibody on the surface of a B cell binds to an antigen, the cell can be stimulated to undergo a process called clonal selection. This process includes proliferation and differentiation, where the cells produced make the same antibody, but become memory cells and plasma cells. Memory cells insure that subsequent infections by that particular pathogen receive a quicker and more efficient response. Plasma cells secrete large quantities of the antigen-specific antibody. The antigen-specific antibody forms complexes with free pathogens and their harmful products, inactivating pathogens and stimulating other innate systems including phagocytic cells and complement to eliminate the danger from extracellular fluids.

The cellular system responds to cells containing pathogen display antigen fragments on their cell surfaces. Receptors on the surface of CD8 cells (cytotoxic T cells) can detect the presence of pathogen specific antigen fragments and activate a killing response known as apoptosis that leads to the death of the infected cell. CD8 cells must interact with CD4 cells (helper T cells) to regulate destruction of infected cells. CD4 cells regulate other cells of the immune system through secretion of molecules called cytokines. Furthermore, CD4 cells are generally required for the clonal selection of B cells, as described above.

CD40 ligand (CD40L, also referred to as CD154, and gp39 (glycoprotein of 39 kiloDaltons) is a critical effector molecule expressed mainly by activated CD4+ T cells and is essential for thymus (T)-dependent immunity. Surface CD40L expressed by activated T cells is required to activate B cells and monocytes via CD40 molecules expressed on those cells. Humans and mice that lack CD40L function suffer from the X-linked form of hyper immunoglobulin M (IgM) syndrome, a severe immune deficiency disease characterized by pyogenic bacterial infections due to the inability of B cells to produce affinity matured antibodies and isotypes other than IgM, and by opportunistic infections due to defective macrophage activation.

Conversely, abnormal CD40L expression by T cells is associated with multiple human immunological and inflammatory diseases such as, but not limited to, systemic lupus erythematosus (SLE) and rheumatoid arthritis (RA). CD40L is also expressed by other cells, such as, for example, endothelial cells and platelets, where it is thought to have a role in inflammation and hemostasis.

Given the relationship of CD40L to human disease and the importance of its controlled expression, knowledge of the transcription factors that regulate CD40L expression in vivo is critically important for understanding normal immune regulation. Accordingly, there exists a need for techniques to manipulate CD40L expression as a way of treating human disorders in which such CD40L expression is abnormal, and yet does not suffer from one or more of the problems exhibited by conventional treatment methodologies.

### SUMMARY OF THE INVENTION

Principles of the present invention provide techniques for treating immune dysfunction by regulation of CD40 Ligand expression.

For example, in one aspect of the invention, a technique treating at least one of an immunological disease and an inflammatory disease in a patient includes the step of suppressing at least one of transcription factor binding to μ enhancer site 3 (TFE3) and transcription factor EB (TFEB) in the patient to thereby suppress CD40L expression. TFE3 and TFEB may be suppressed, in accordance with a preferred embodiment of the invention, by blocking the synthesis of at least one of TFE3 and TFEB, and/or by blocking an ability of the TFE3 and TFEB molecules to interact with each other and/or interact (for example, bond) with deoxyribonucleic acid (DNA). Shown in the drawings that support the present invention are the effects of two types of TFE3/TFEB inhibitors on CD40L expression, a trans-dominant negative protein (TDN) and stem-loop RNA mediated interference (slRNA).

These and other features, objects and advantages of the present invention will become apparent from the following detailed description of illustrative embodiments thereof, which is to be read in connection with the accompanying drawings.

### BRIEF DESCRIPTION OF THE DRAWINGS

FIG. 1a depicts an illustrative immunoblot of TFE3 and TFEB in primary CD4+ mouse splenocytes and human Jurkat T cells, according to an embodiment of the present invention;

FIG. 1b depicts an illustrative time course of TFE3 and TFEB protein expression in mouse CD4 T cells after TCR stimulation, according to an embodiment of the present invention;

FIG. 1c depicts an illustrative analysis of Mitf protein expression in mouse CD4 and human Jurkat T cells before and after stimulation and in mouse thymocytes, according to an embodiment of the present invention;

FIG. 1d depicts an illustrative semi-quantitative reverse transcriptase dependent-polymerase chain reaction (RT-PCR) assay to measure relative abundance of Tcfe3, Tcfeb, and Mitf RNA transcripts in resting and stimulated CD4+ T cells over a time course, according to an embodiment of the present invention;

FIG. 1e is an illustrative schematic view depicting the EμPμ-TDN (“Eμ” and “Pμ” refer to the immunoglobulin μ heavy chain intronic enhancer and promoter, respectively) transgene vector used in one or more embodiments of the present invention;

FIG. 1f depicts an illustrative immunoblot of TDN protein in extracts from total bone marrow (BM), total spleen (Spleen) and total thymocytes (Thymus) from trans-dominant negative-transgenic (TDN-Tg) mice (+) but not in the same types of cells isolated from non-transgenic (non-Tg) littermates (−), according to an embodiment of the present invention;

FIG. 1g depicts illustrative flow cytometry histograms of permeabilized lymphocytes from non-Tg and the T cell-specific TDN-Tg mice for intensity of staining with an anti-hemagglutinin (-HA) antibody that detects the TDN protein, according to an embodiment of the present invention;

FIG. 1h depicts an illustrative immunoblot of TDN protein present in extracts of purified CD4+ T cells, but not splenic B cells, from TDN-transgenic mice, according to an embodiment of the present invention;

FIGS. 2a and 2b are illustrative graphical views of cell counts and flow cytometry depicting how numbers and a distribution of major B and T cell populations is normal in TDN-transgenic mice, according to an embodiment of the present invention;

FIGS. 3a and 3b are illustrative graphical views using immunohistochemistry and flow cytometry to depict impaired germinal center formation in TDN-transgenic mice, according to an embodiment of the present invention;

FIGS. 4a through 4d are illustrative graphical views of ELISA assays depicting impaired T-dependent, but normal T-independent, humoral responses in TDN-transgenic mice, according to an embodiment of the present invention;

FIG. 5a depicts an illustrative flow cytometry demonstrating impaired surface expression of CD40L by TDN-transgenic (TDN) as compared to non-Tg control (wild type (WT)) CD4+ T splenocytes stimulated with monoclonal antibodies (mAb) to CD3 and analyzed at 8 hours, according to an embodiment of the present invention;

FIG. 5b depicts an illustrative quantification of flow cytometry histogram data for percent CD4+ T cells positive for CD40L (left) and CD25 (right) after 8 hours of stimulation with various amounts of mAb to CD3 (Anti-CD3), according to an embodiment of the invention;

FIGS. 5c and 5d depict illustrative flow cytometry demonstrating comparable levels of ICOS (inducible Co-stimulator of T cells) and CD28, respectively, by control (wild type (wt)) and TDN-transgenic (TDN) CD4+ splenocytes, according to an embodiment of the present invention;

FIG. 5e depicts an illustrative graphical representation of ELISA (enzyme linked immunoadsorbance assay) analyses showing comparable levels of interleukin-4 (IL-4) secretion by control (wild type (WT)) and TDN-transgenic (TDN) CD4+ splenocytes, according to an embodiment of the present invention;

FIG. 5f depicts an illustrative bar graph showing normal proliferation of TDN-Tg T cells in response to CD3/CD28 stimulation, according to an embodiment of the present invention;

FIG. 5g depicts a representative semi-quantitative RT-PCR analysis showing that Smad 7 expression is normal in TDN-Tg splenic T cells, according to an embodiment of the invention;

FIG. 5h depicts an illustration of representative flow cytometry histograms depicting impaired CD40L but normal CD25 expression by TFE3-deficient (Tcfe3−/−) CD4 T cells infected with a lentivirus that expresses an interfering stem-loop RNA against TFEB, according to an embodiment of the present invention;

FIG. 5i depicts an illustrative representation of an immunoblot showing the expected down-regulation of TFEB protein in CD4+ T cells infected with a retrovirus that expresses the interfering stem-loop TFEB RNA in support of the data in FIG. 5h, according to an embodiment of the present invention;

FIG. 5j depicts an illustrative real-time RT-PCR analysis of ribonucleic acid (RNA) from CD4+ T cells showing reduced abundance of CD40lg transcripts in CD4+ T cells from TDN-transgenic mice relative to non-transgenic littermates at various times after incubation with mAb to CD3 (horizontal axis), according to an embodiment of the invention;

FIG. 6a depicts an illustrative flow cytometry histogram demonstrating indistinguishable CD40 expression in B cells from non-transgenic and TDN-transgenic mice, according to an embodiment of the present invention;

FIGS. 6b and 6c depict illustrative flow cytometry histograms, demonstrating equivalent expressions of CD86 and major histocompatibility complex (MHC) class II, respectively, after stimulation of B cells from non-transgenic and TDN-transgenic mice with mAb to CD40, according to an embodiment of the present invention;

FIG. 6d depicts an illustrative ELISA measuring immunoglobulin G (IgG) responses to TNP-KLH (Trinitrophenol-keyhole limpet hemocyanin) which shows equivalent responses in non-transgenic and TDN-transgenic mice treated with an agonist mAb to CD40 during immunization, according to an embodiment of the present invention;

FIG. 7a depicts an illustrative schematic alignment of promoters for the gene encoding CD40L from humans, mice and rats, showing MiT (the Mitf/TFE family) sites (a subset of E-boxes; “E” refers to “enhancer” boxes from the IgH enhancer) where TFE3 and TFEB bind to and activate the CD40lg and CD40LG promoters, according to an embodiment of the present invention

FIG. 7b depicts an illustrative Chromatin immunoprecipitation (ChIP) of a CD40lg promoter fragment by anti-TFE3 and anti-TFEB antibodies, as well as the illustrative controls for the ChIP assays, according to an embodiment of the present invention;

FIG. 7c depicts illustrative electrophoretic mobility-shift assays (EMSA) demonstrating the binding of native TFE3 and TFEB proteins present in CD4 T cell extracts to individual MiT sites from the Cd40lg promoter, according to an embodiment of the present invention;

FIG. 7d depicts illustrative electrophoretic mobility shift assays (EMSAs) of nuclear extracts of human embryonic kidney (HEK293) cells transfected with control (−), TFE3, or TFFB pFBB expression vectors showing TFE3 and TFEB binding to the MiT sites from the Cd40lg promoter, according to an embodiment of the present invention;

FIG. 8a is a bar graph depicting illustrative luciferase assays to measure Cd40lg promoter activity in CD4 T cells from non-Tg and TDN-Tg mice, showing that a CD40lg fragment containing the MiT sites depends on endogenous TFE3 and TFEB to enhance promoter activity, according to an embodiment of the present invention;

FIG. 8b is a bar graph depicting illustrative luciferase assays showing the contributions of individual and combinations of MiT sites to Cd40lg promoter activity in non-Tg, TDN-Tg, and Tcfe3−/− CD4 T cells, according to an embodiment of the present invention;

FIG. 8c depicts immunological assays that show that native TFE3 and TFEB in CD4 T cells can form heterodimers, an interaction that is blocked by the TDN protein, according to an embodiment of the present invention;

FIG. 9a is a bar graph depicting illustrative luciferase assay showing the importance of MiT sites and endogenous TFE3 and TFEB for the human CD40LG and mouse CD40lg promoter activity in human Jurkat T cells, according to an embodiment of the present invention;

FIG. 9b is an illustrative graphical view of a flow cytometry histogram depicting how inhibition of TFE3 and TFEB activity by expression of the TDN protein blocks endogenous CD40L induction after activation of human Jurkat T cells but that CD25 induction is not affected, according to an embodiment of the present invention;

FIG. 10a depicts a table illustrating oligonucleotides for EMSA in FIGS. 7, including SEQ. ID NOs: 1-16, according to an embodiment of the present invention;

FIG. 10b depicts a table illustrating oligonucleotides for cloning Cd40lg and CD40LG promoters, including SEQ. ID NOs: 17-22, according to an embodiment of the present invention;

FIG. 10c depicts a table illustrating oligonucleotides for E-box mutagenesis, including SEQ. ID NOs: 23-30, according to an embodiment of the present invention;

FIG. 11a depicts an immunoblot showing that TFE3 and TFEB proteins are expressed in B cells, according to an embodiment of the present invention;

FIG. 11b depicts a representative immunoblot showing TDN protein in B cell containing lymphoid organs from B-cell-specific TDN-Tg mice, according to an embodiment of the present invention;

FIG. 11c depicts representative flow cytometry histograms showing that the TDN protein is expressed only in B, but not T cells in “B-cell-specific TDN-Tg” mice, according to an embodiment of the present invention;

FIG. 11d depicts representative flow cytometry dot plots showing that major B and T cell populations in these “B cell-specific TDN-Tg” mice are normal according to an embodiment of the present invention; and

FIG. 12 is a flow chart illustrating a technique for treating at least one of an immunological disease and an inflammatory disease in a patient, according to an embodiment of the present invention.

### DETAILED DESCRIPTION OF THE INVENTION

One or more embodiments of the present invention meet the above-noted need by providing methods of treating an immune disease in which CD40L expression is abnormal or otherwise drives pathology. The invention, in an illustrative embodiment thereof, beneficially exploits an advantageous role of transcription factor binding to immunoglobulin heavy chain μ (IGHM) enhancer (μE) site 3 (TFE3) and transcription factor EB (TFEB) in regulating CD40L expression. One or more embodiments of the present invention teach, for example, that TFE3 and TFEB directly activate CD40L at the transcriptional level in vivo without significantly affecting the synthesis of other activation and effector molecules. Inactivation of TFE3 and TFEB attenuates the induction of CD40L expression on T cells.

TFE3 and TFEB are broadly expressed transcription factors related to the transcription factor Mitf. Although they have been linked to cytokine signaling pathways in non-lymphoid cells, their function in T cells is unknown. TFE3-deficient mice are phenotypically normal, whereas TFEB deficiency causes early embryonic death. One or more embodiments of the present invention illustrate that combined inactivity of TFE3 and TFEB in T cells result in a hyper-immunoglobulin M (hyper-IgM) syndrome due to impaired expression of the CD40 ligand gene (Cd40lg) by CD4+ T cells.

As previously stated, abnormal CD40L expression by T cells is associated with multiple human immunological and inflammatory diseases such as, but not limited to, systemic lupus erythematosus (SLE) and rheumatoid arthritis (RA). CD40L is also expressed by other cells, such as, for example, endothelial cells and platelets, where it is thought to have a role in inflammation and hemostasis.

As used herein, “CD40L” refers to the protein, “Cd40lg” refers to the name of the mouse gene encoding the CD40L protein, and “CD40LG” refers to the human gene. Similarly, TFE3 and TFEB refer to the proteins, whereas Tcfe3 and Tcfeb refer to the mouse genes.

Native TFE3 and TFEB bound to multiple cognate sites in the promoter of the gene encoding CD40 ligand, and maximum Cd40lg promoter activity and gene expression required TFE3 or TFEB. Consequently, as illustrated by one or more embodiments of the invention, TFE3 and TFEB are direct, physiological and mutually redundant activators of Cd40lg expression in activated CD4+ T cells critical for T cell-dependent antibody responses.

The teachings of one or more embodiments of the present invention relate generally to the regulation of CD40L expression. Control of CD40L expression by T cells is complex and highly regulated, befitting its central role in immune activation and modulation. Although one or more embodiments of the present invention is described herein in the context of T cells and B cells, it is contemplated that the techniques of the invention may be used for controlling CD40L expression by immune cells in general. The phrase “immune cells” as used herein is intended to include (but is not limited to) hematopoietic and endothelial cells.

CD40L is rapidly and transiently induced upon stimulation of naïve T cells via the T cell receptor (TCR), with surface levels peaking by 6-8 hours and then declining. This profile of TCR-dependent CD40L induction primarily reflects transcriptional activity. CD40L expression can be prolonged and augmented by additional stimulatory input from co-stimulatory and/or accessory molecules such as, but not limited to, CD28, CD2, LFA-1 (lymphocyte-function associated antigen-1), and CD43, and interleukins, such as IL-2, IL-12, and IL-15, via mechanisms that involve enhanced transcription, increased CD40L messenger-RNA (mRNA) stability, or both. Consequently, the overall dynamics of CD40L expression is dependent on TCR stimulatory conditions, activation and/or effector status, developmental stage of the T cell, and age.

Mice engineered to express CD40L via heterologous promoters in T cells have been shown to develop lymphoproliferative disorders and tumors, highlighting the importance of regulated CD40L transcription (see, for example, Brown, M. P. et al., “Thymic lymphoproliferative disease after successful correction of CD40 ligand deficiency by gene transfer in mice,” Nat Med 4, pp. 1253-1260 (1998), and Sacco, M. G. et al., “Lymphoid abnormalities in CD40 ligand transgenic mice suggest the need for tight regulation in gene therapy approaches to hyper immunoglobulin M (IgM) syndrome,” Cancer Gene Ther. 7, pp. 1299-1306 (2000), the disclosures of which are incorporated by reference herein).

Multiple transcription factors and cognate promoter and enhancer regulatory elements responsive to the TCR and co-stimulatory receptor stimulation have been identified that regulate the transcription of CD40L. Like many effector and activation molecules induced by T cell stimulation, such as, for example, IL-2 and tumor necrosis factor (TNF) family genes, CD40L induction is dependent on subunits of nuclear factors of activated T-cells (NFAT), a Ca²⁺-dependent transcription factor complex. Additional regulators include subunits of AP-1 (Activator Protein-1) and NFkB (Nuclear Factor that binds to the kappa enhancer B site) and Egr-1 (early growth response-1), which were also shared by other TCR and/or co-stimulator responsive genes, and AKNA. Binding sites for other transcription factors, such as, for example, STAT5, GATA-3, Oct-1 subunits, have also been identified (see, for example, Crow, M. K. & Kirou, K. A, “Regulation of CD40 ligand expression in systemic lupus erythematosus,” Curr Opin Rheumatol 13, pp. 361-369 (2001), and Cron, R. Q, “CD154 transcriptional regulation in primary human CD4 T cells,” Immunol Res 27, pp. 185-202 (2003), which are incorporated by reference herein), but their physiological contribution to CD40L transcription is not yet known.

Despite these commonalities, the dynamics of CD40L expression and its responsiveness to various stimuli are distinct from other molecules induced by T cell activation and therefore suggest that the regulation of CD40L transcription in T cells may involve as yet unknown factors or elements. Similarly, relatively less is known about how CD40L transcription is regulated in non-lymphoid cells.

Given the relationship of CD40L to human disease and the importance of its controlled expression, knowledge of the transcription factors that regulate CD40L expression in vivo is critically important for understanding normal immune regulation and for designing strategies to manipulate CD40L expression as a way to treat the multiple human disorders in which CD40L expression is abnormal or undesirable.

In accordance with one embodiment of the present invention, the transcription factors TFE3 and TFEB are demonstrated to be physiological transcription regulators of CD40L expression in T cells. TFE3 is a member of the helix-loop-helix family of transcription factors that binds to the mu-E3 (μE3) motif of the immunoglobulin heavy-chain enhancer and is expressed in many cell types. TFEB is a transcription factor with a basic region-DNA binding domain, a helix-loop-helix and leucine zipper dimerization domains, and a nuclear localization signal, thought to be located adjacent to the helix-loop-helix domain. TFEB is ubiquitously expressed.

TFE3 and TFEB are closely related members of a functionally interactive DNA binding family known as Mitf/TFE (MiT), that includes the microphthalmia transcription factor Mitf and TFEC (Transcription factor E-box C). MiT proteins bind to so called μE3 sites, a subset of E-boxes that match a general CANNTG consensus sequence, with those binding to TFE3 in vitro first identified and characterized in immunoglobulin heavy-chain and T cell receptor (TCR) enhancers. DNA binding is mediated by nearly identical basic regions (BRs) and requires dimer (either homodimer or heterodimer) formation between MiT family members mediated by conserved helix-loop-helix (HLH) and leucine zipper (LZ) domains.

MiT proteins share similar structures and are often expressed together, yet genetic studies have demonstrated both overlapping and non-overlapping functions for MiT proteins in different cell-types. Mitf, a well-characterized family member, is expressed mainly in pigment and myeloid cells, where it is involved in melanocyte and mast cell development as a transcription mediator of the c-kit pathway, and is a negative regulator of B cell activation and terminal differentiation. TFE3 and Mitf serve redundant roles in osteoclast development as transcriptional mediators of the macrophage colony-stimulating factor (m-csf) pathway. These functions of Mitf correspond to its cell-type and/or lineage restricted expression pattern.

Like expression of Mitf, TFEC expression is restricted mainly to the myeloid lineage, but TFEC-deficient mice are phenotypically normal, even though TFEC-deficient macrophages have lower expression of a subset of interleukin 4 (IL-4)-responsive transcripts, including the transcript for granulocyte-macrophage colony-stimulating factor.

In contrast, TFE3 and TFEB are more broadly expressed, and relatively less is understood about their individual biological functions, primarily because germline TFE3-deficient animals have no reported defects. Studies of cultured non-lymphoid cell lines have indicated that TFE3 facilitates the activation of a subset of genes dependent on transcription factor Smad3 (Mothers against decapentaplegic homolog 3), in response to transforming growth factor-β) (TGF-β)) including those encoding components of the extracellular matrix and Smad7, a TGF-β pathway inhibitor. Ectopic over-expression of TFE3 in hepatocytes in mice promotes glycogen synthesis. However, mice deficient in TFE3 are phenotypically normal, with no defects noted in development, reproduction or the immune response. In contrast, TFEB deficient embryos die early in gestation because of defects in placental vascularization. The function of TFEB in adults is not known.

Given the extensive amino acid sequence similarities and overlapping expression profiles, a possible explanation for the former observation (that is, that Tcfe3−/− mice are normal) is that TFEB and TFE3 are functionally redundant in many tissues and, to varying degrees, TFEB may compensate for a TFE3-deficiency. By selectively inactivating these TFE3 and TFEB molecules, the present invention, in accordance with an illustrative embodiment thereof, exploits a central role of TFE3 and TFEB in the immune system via their control of CD40L expression, and thereby provides a beneficial methodology for treating immune dysfunction. The phrase “immune dysfunction” as used herein is intended to refer to any syndrome in which activity of immunocytes is undesirable, including, but not limited to, leukemia, lymphoma, lupus and rheumatoid arthritis, transplant rejection, asthma and other allergic diseases, autoimmune diseases, etc.

One or more embodiments of the present invention demonstrate that TFE3 and TFEB are critical for T cell function and humoral immunity through their direct control of CD40L expression. This was demonstrated by expression of the TDN protein in mouse T cells in vivo or in human T cells in culture, which blocked existing TFE3 and TFEB proteins expressed therein, and by de novo inhibition of TFEB protein synthesis via expression of an interfering RNA against TFEB in Tcfe3−/− T cells. TDN expression in mouse T cells in vivo via the EμPμ transgene resulted in hyper-IgM syndrome caused by defective CD40L expression.

As mentioned previously, like the induction of many effector and activation molecules by T cell stimulation, such as IL-2 and TNF family members, CD40L induction is dependent on subunits of NFAT, a calcium-responsive transcription factor complex. Additional regulators include the AT-Hook (“AT-Hook” refers to the AT-rich sequences this type of transcription factor binds to and the shape of the protein itself) transcription factor AKNA and subunits of transcription factors AP-1, NF-kB and Egr-1, which are also shared by other TCR and/or co-stimulator-responsive genes. However, unlike the aforementioned genes, Cd40lg is the only one identified that requires TFE3 or TFEB. That requirement may contribute to the unique expression profile of Cd40lg compared with that of other TCR-responsive genes. Identifying the conditions and pathways that control TFE3 and TFEB activity is therefore important in understanding the control of CD40L-dependent immunity and immunopathology.

In activated CD4+ T cells, TFE3 and TFEB were mutually redundant in controlling Cd40lg transcription, as a combined deficiency in TFE3 and TFEB inhibited TCR-dependent CD40L induction in the primary T cell culture systems, whereas individual deficiencies had a minor or no effect. Thus, one or more embodiments of the present invention have demonstrated physiological functional redundancy between TFE3 and TFEB in vivo, which may be a more general property of these proteins in other cell types. A degree of functional redundancy has also been reported among NFAT components in activating Cd40lg transcription, as a combined deficiency of NFATc1 and NFATc2 (individual members of the NFAT family, respectively) results in greater impairment in CLD40L expression than does individual deficiency.

However, it remains possible that, in certain conditions, TFE3 and TFEB each have a unique contribution to the activation of Cd40lg. For example, only TFEB protein was induced after TCR stimulation of primary cells, most likely through post-transcriptional mechanisms, and TFEB bound to the Cd40lg promoter independently of TFE3. There were also differences in the relative importance of a subset of E-boxes to Cd40lg promoter activity in wild-type versus Tcfe3−/− (Tcfe3 refers to the gene encoding TFE3 T cells. Consequently, there may be physiological contexts in which TFEB or TFE3 may be more advantageous, such as, for example, for NFAT subunits in T cell subsets or in other cell types expressing CD40L.

The very low, rather than absent, CD40L expression in activated T cells lacking TFE3 and TFEB activity is consistent with the limited degree of germinal center formation in TDN-transgenic mice, in contrast to that of mice with genetic Cd40lg deficiency, in which no germinal centers are found. We believe that the remaining CD40L expression was not due to incomplete inactivation of TFE3 and TFEB by the TDN protein, given that TDN protein expression inhibited the binding of endogenous TFE3 or TFEB to the Cd40lg promoter to basal binding amounts, as evaluated by electrophoretic mobility-shift assay and chromatin immunoprecipitation. It is, however, possible that other μE3 (also referred to as “MiT”) site-binding transcription factors not in the MiT family may bind to the promoter in their absence. These may include upstream stimulating factor (USF-1) and c-Myc, which are from distinct and non-interactive helix-loop-helix families.

USF-1 binds to a promoter target site normally occupied by TFE3 in Tcfe3−/− but not wild-type fibroblasts. It has been suggested that c-Myc can also activate the Cd40lg promoter, but its effect is apparently indirect. Yet regardless of MiT site occupancy, we postulate that the absence of TFE3 and TFEB raises the activation threshold of the Cd40lg promoter by the remaining activators, such as NFAT and AP1. In that scenario, very large and sustained amounts of the other transcription activators, coupled with synergistic mechanisms such as mRNA and protein stabilization, could allow sufficient CD40L accumulation to overcome the TFE3 and TEEB deficiency in certain T cell clones highly activated by TCR, co-stimulators and interleukins.

The normal distribution of the main T cell populations in non-immunized TDN-transgenic mice suggested that other possible MiT target genes in T cells were not required for T cell development itself beyond the double-positive stage when the TDN protein was first detected. Those findings are consistent with the reported lack of developmental defects of Mitf^{Mi/Mi} (Mitf refers to the gene encoding the Mitf protein, and the Mi/Mi refers to the particular mutant allele of Mitf) T cells and indicate that TFE3 and TFEB are also not essential.

Also unaffected were the expression of several TCR-responsive genes other than Cd40lg, and antibody responses to the thymus-independent antigen. Those data suggest that MiT deficiency caused a relatively restricted T cell defect, in contrast to deficiencies in NFAT and NF-B subunits, which can radically affect T cell development, effector function or homeostasis.

Given those observations, we believe a deficit in CD40L expression is sufficient to account for the impaired T cell-dependent antibody responses noted in TDN-transgenic mice. Further, in some humans with X-linked hyper-IgM syndrome, the genetic defect leads to low rather than no or mutant CD40L expression. Nevertheless, one or more embodiments of the invention do not rule out the importance of other putative TFE3 and TFEB target genes in T helper-cell finction not specifically addressed herein, or their importance in positive and negative selection. Although TFE3 has been directly and indirectly linked to several cytokine signaling pathways that control cell growth and differentiation in non-lymphoid cells, including c-Kit (c-kit is a tyrosine kinase receptor that is mutated in malignancies and is activated by “steel factor,” and is also referred to as the c-kit ligand. Another name for c-kit is CD117. C-kit is critical for the development of mast cells and melanocytes) and transforming growth factor-β, cell type-specific and gene-specific, regulation by each MiT protein is an established characteristic of this family. Pathway involvement established in one cell type cannot be directly extrapolated to other cell types and must be determined experimentally.

Many human diseases are associated with abnormal CD40L expression. For example, CD40L expression is often constitutively increased on T cells from patients with systemic lupus erthythematosus and rheumatoid arthritis. In systemic lupus erthythematosus, this occurs without abnormal expression of other markers of T cell activation. Transcriptional and post-transcriptional mechanisms that normally enhance CD40L expression are all thought to contribute to that phenomenon.

It has been shown that the Ras GTPase-mitogen-activated protein kinase (MAPK) pathway is necessary for the maintenance of abnormal CD40L expression by T cells from patients with systemic lupus erthythematosus. Given that in non-lymphoid cells, the transcriptional activity of TFE3 and TFEB can each be regulated by the Ras-mitogen-activated protein kinase (-MAPK) pathway, for example, in response to activation of the m-csf receptor, a key issue will be whether the activity of TFE3 and TFEB can be regulated by MAPK activation in T cells (for example, in response to the TCR and IL-15 receptor) and if there are differences in their activity in normal versus autoimmune T cells. MAPK activation of TFE3 and TFEB can be defined, at least in part, as phosphorylation which enables TEF3 and TEFB to interact with other molecules critical for transcription.

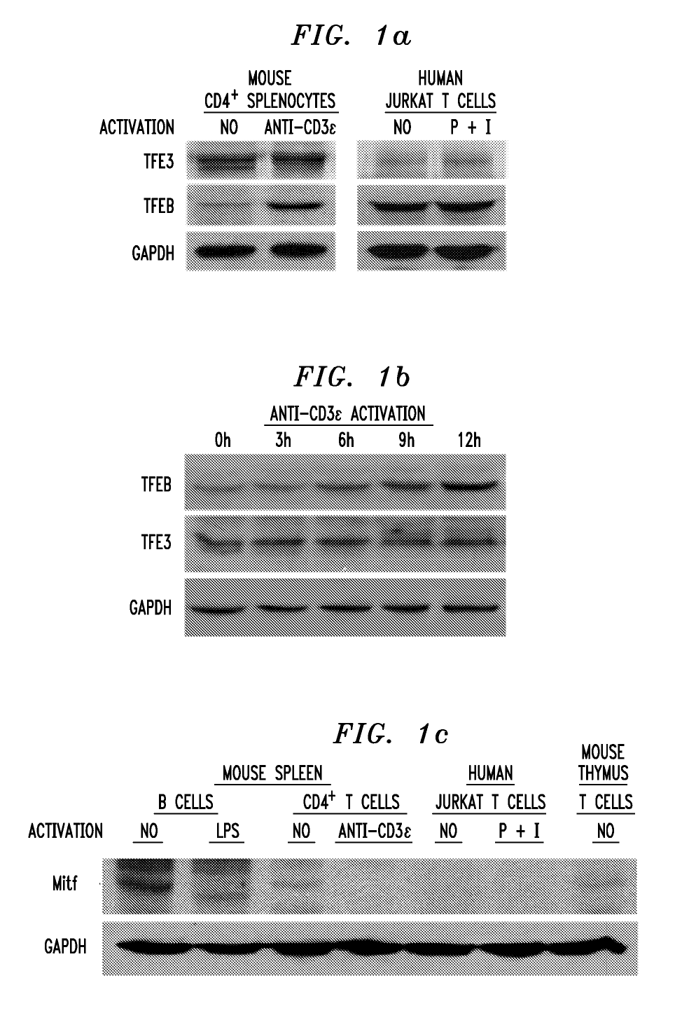

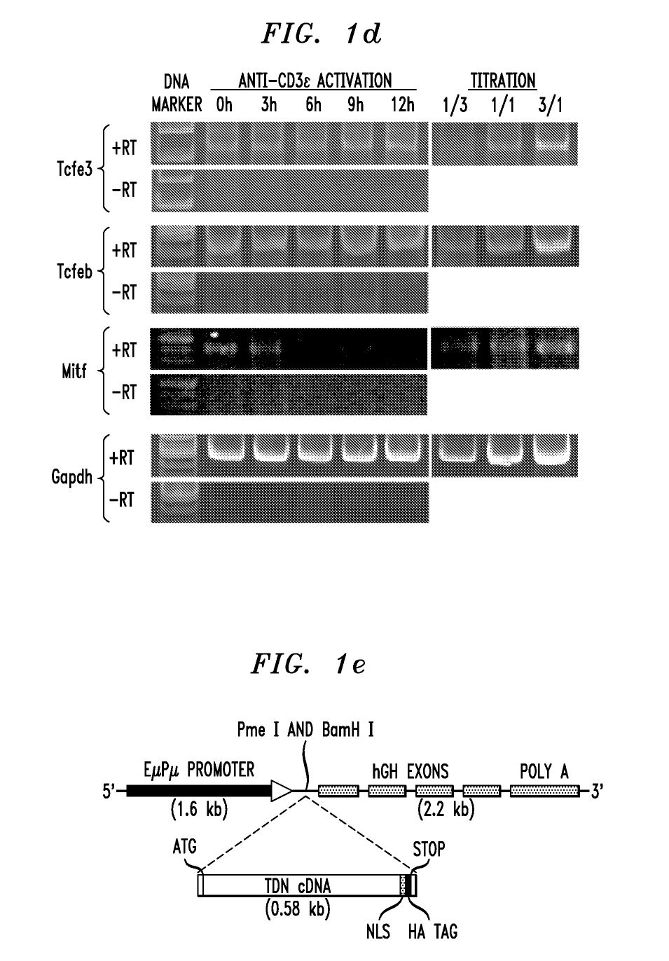

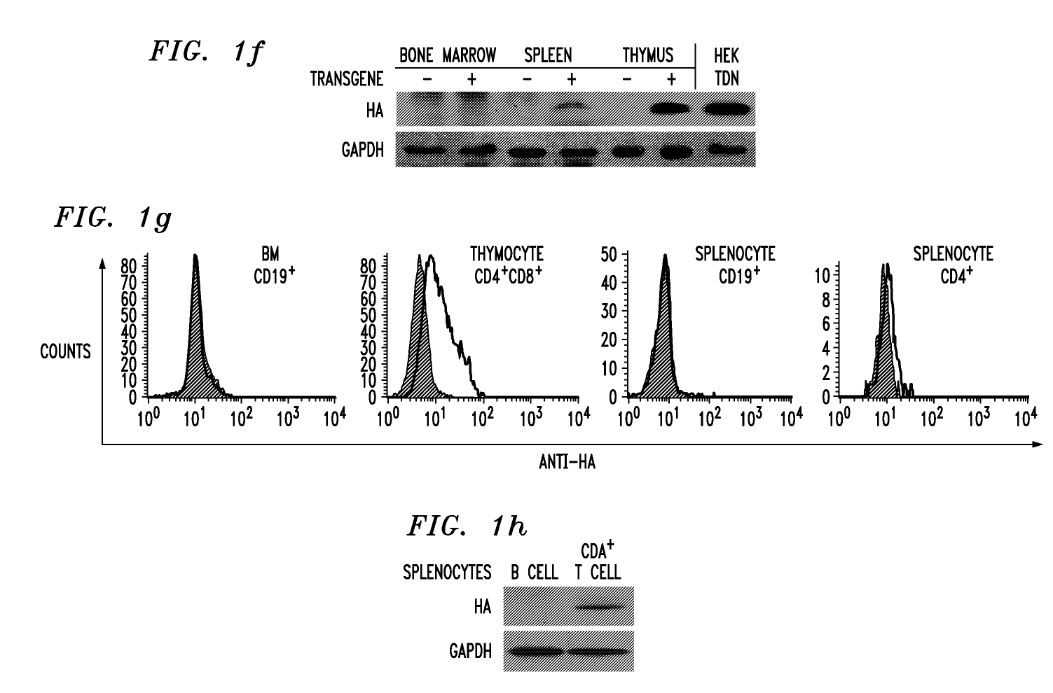

FIG. 1a depicts an illustrative immunoblot of TFE3 and TFEB protein in un-stimulated and stimulated primary CD4+ mouse splenocytes (via CD3 mAb) and human Jurkat T cells (stimulated via phorbol myristate acetate [PMA] plus ionomycin), according to an embodiment of the present invention. The GAPDH immunoblot is also shown as a loading control.

FIG. 1b depicts an illustrative time course of TFE3 and TFEB protein expression after TCR stimulation, according to an embodiment of the present invention. CD4+ T cells were stimulated with the CD3 mAb and aliquots taken at each time point for analysis by immunoblot. These data are extensions of the data in FIG. 1a.

FIG. 1c depicts an illustrative analysis of Mitf protein expression, according to an embodiment of the present invention. Extracts from resting and LPS stimulated CD19+ B cells, resting and CD3-stimulated (8 hours) CD4+ T cells, un-stimulated and pharmacologically stimulated Jurkat T cells, and resting thymocytes, were probed by immunoblot with a Mitf mAb. The cross-reactive band corresponding in size to the A isoform of Mitf is indicated with the line extending from “Mitf.” This designation is also based on the disappearance of this band in stimulated B cells. The GAPDH immunoblot is also shown as a loading control.

FIG. 1d depicts an illustrative semi-quantitative reverse transcriptase (RT) dependent-polymerase chain reaction (PCR) to measure relative abundance of Tcfe3, Tcfetb, (the mouse genes encoding TFE3 and TFEB proteins, respectively) and Mitf mRNA transcripts in resting and stimulated CD4+ T cells over a time course, according to an embodiment of the present invention. Reverse transcriptase, in this context, is required to convert the mRNA into a DNA copy (cDNA) that is then used as a template for amplification in the PCR reaction. The identities of the bands are indicated. The titrations illustrate the relative band intensities of PCRs programmed with the same amount of 0 hour complementary DNA (cDNA) used for the PCR series (1/1), one-third the amount (1/3), or three-fold more (3/1).

Immunoblot (FIGS. 1a, 1b, and 1c) and RT-PCR (FIG. 1d) analyses were used to assess the MiT protein and mRNA expression profile, respectively, in T cells. Of the four MiT proteins, only TFE3 and TFEB proteins were detected in CD4+ splenic mouse T cells and in the human transformed T cell line Jurkat, as shown in FIG. 1a, which depicts an exemplary immunoblot of TFE3 and TFEB in primary CD4+ mouse splenocytes and Jurkat T cells. With reference to FIGS. 1a, 1b, 1c, and 1d, byway of example only, CD4+ splenic T cells were stimulated by incubation with an anti-CD3 mAb for 8 hrs. Jurkat T cells were stimulated with phorbol myristate acetate (PMA)/ionomycin (Iono) for 20 hours. Glyseraldehyde-3-phosphate dehydrogenase (GAPDH) protein was the loading control. The TFEB and GAPDH antibodies recognized epitopes on the corresponding proteins from mouse and human and were used for probing both extracts. However, TFE3 proteins from mouse and human extracts were revealed with species restricted anti-TFE3 antibodies. Consequently, band intensities cannot be directly compared between mouse and human. The exemplary images shown in FIGS. 1a-1d are representative of at least three independent experiments.

Notably, TFEB protein levels increased (five-fold) in response to TCR-engagement in parallel with CD40L induction in primary mouse T cells, whereas TFEB levels were already elevated in un-stimulated Jurkat cells and did not change in response to stimulation. In contrast, TFE3 levels remained relatively constant in both cases, as apparent from FIG. 1a. The increase in TFEB was at the post-transcriptional level, as relative amounts of steady-state Tcfeb (the mouse gene encoding TFEB protein) mRNA were unchanged (FIG. 1d). A band was detected that was interpreted to be the A isoform of Mitf in un-stimulated CD4+ T cells, but we did not detect it in TCR-stimulated cells (FIG. 1c). This corresponds to a decrease in Mitf mRNA (FIG. 1d).

We did not detect TFEC expression in any of these samples by either immunoblot or RT-PCR (data not shown). In the human transformed T cell line Jurkat, both TFE3 and TFEB proteins were present and their abundance did not change in response to pharmacological stimulation (FIG. 1a). We did not detect Mitf or TFEC in these cells (FIG. 1c; TFEC data not shown). Thus, in TCR-activated (via CD3 mAb) mouse CD4+ T cells and pharmacologically activated (via PMA/iono) Jurkat T cells, TFE3 and TFEB were the only MiT family members expressed.

One or more embodiments of the present invention illustrate that T-cell-specific inactivation of endogenous TFE3 and TFEB in vivo via transgenesis results in a phenotype resembling X-linked hyper-IgM syndrome.

FIG. 1e is an illustrative schematic view depicting an EμPμ-TDN (“Eμ” and “Pμ” refer to the immunoglobulin μ heavy chain intronic enhancer and promoter, respectively) transgene vector used to create the TDN-transgenic (Tg) mice, according to an embodiment of the present invention. The construction of the TDN protein has been described (see, for example, Huan, C., Sashital, D., Hailemariam, T., Kelly, M. L. & Roman, C. A. J., Renal carcinoma associated transcription factors TFE3 and TFEB are leukemia-inhibitory factor-responsive transcription activators of E-cadherin,” J Biol Chem 13, p. 13 (2005), which is incorporated by reference herein). It contains the HLH-Z region from TFE3 but lacks the DNA binding BR and transcriptional activation domains. A SV40-nuclear localization signal was added to compensate for the loss of that activity by deletion of the BR, and a HA-tag was added for immunodetection.

FIG. 1f depicts an illustrative immunoblot of extracts from total bone marrow (BM), total spleen (Spleen) and total thymocytes (Thymus) from TDN-transgenic mice (+) and non-transgenic littermates (−), according to an embodiment of the present invention. Non-transgenic mice are control littermates that did not receive the chromosome containing the transgene from the transgenic parent. FIG. 1f depicts, in part, TDN expression in total extracts of HEK293 cells transfected with a plasmid expressing the TDN cDNA (lane labeled HEK TDN) for comparison. The anti-HA antibody detects the cognate hemagglutinin (HA) epitope built into the TDN protein. FIG. 1f shows that the TDN protein is only detected in lymphoid organs that contain T cells from TDN-Tg mice and not in non-Tg mice, which established, in part, that TDN expression in this transgenic line was T cell specific.

FIG. 1g depicts illustrative flow cytometry histograms of permeabilized lymphocytes from non-Tg and T cell-specific TDN-Tg mice for staining with an anti-HA antibody that detects the TDN protein, according to an embodiment of the present invention. In the exemplary histograms in FIG. 1g, filled curves are HA staining profiles of non-Tg (control) cells, and line overlays are profiles of cells from TDN-Tg mice from bone marrow, spleen, and thymocytes. B and T lymphocyte populations are delineated using lineage-specific markers.

FIG. 1h depicts an illustrative immunoblot of extracts of purified splenic B cells and CD4+ T cells from TDN-transgenic mice (−), showing that the TDN protein is expressed only by splenic T cells but not in B cells from TDN-Tg mice, according to an embodiment of the present invention. FIG. 1h includes, in part, an immunoblot for the hemagglutinin (HA) epitope built into the TDN protein, and GAPDH, a loading control. Data are representative of at least three independent experiments.

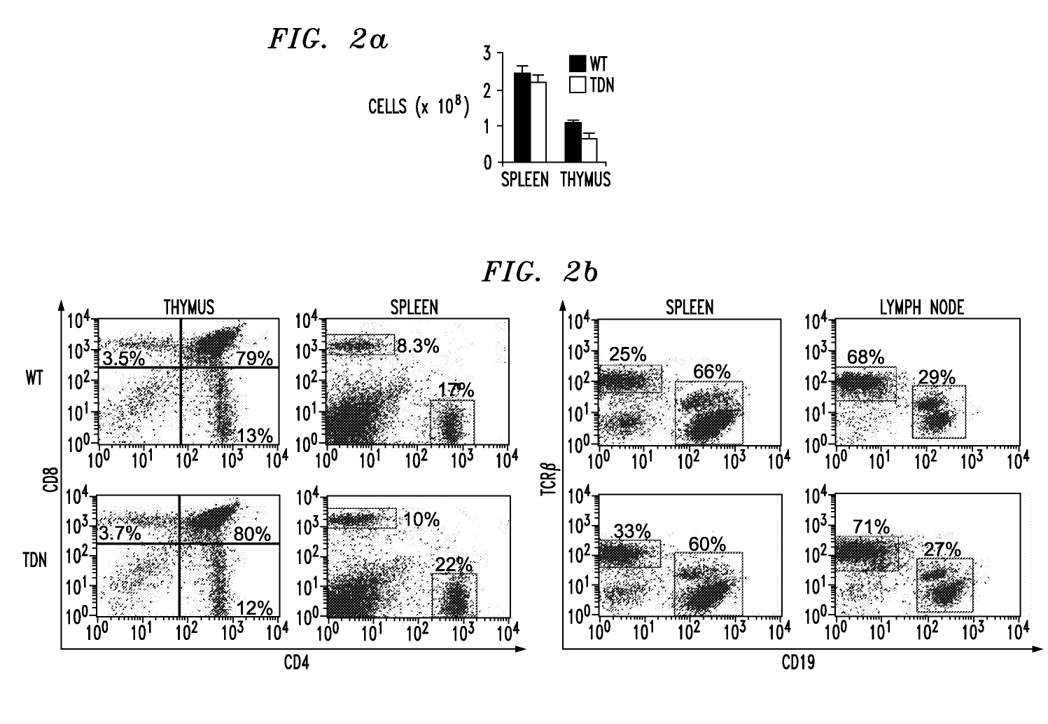

FIGS. 2a and 2b are illustrative graphical views depicting how the numbers and a distribution of major B and T cell populations is normal in TDN-transgenic mice, according to an embodiment of the present invention.

TDN-transgenic mice exhibited humoral immune defects that were consistent with a hyper IgM syndrome caused by a deficit in CD40L expression. An EμPμ transgene, as described, for example, in Tepper, R. I. et al., “IL-4 induces allergic-like inflammatory disease and alters T cell development in transgenic mice,” Cell 62, pp. 457-467 (1990), which is incorporated by reference herein, was used to direct expression of the TDN protein. In one or more embodiments of the present invention, transgenic mouse lines were analyzed in which the TDN was expressed exclusively in T cells, which was shown in FIGS. 1f-1h. TDN expression was found to be highest in thymocytes and lower in mature single positive splenic T cells. No TDN protein was detected in B cells or non-lymphoid cells and tissues (see, for example, FIGS. 1f-1h).

Analysis of lymphoid compartments in TDN-transgenic mice by cell counting and flow cytometry revealed that B and T cell development proceeded, with normal to nearly normal numbers and distributions of major splenic B and T cell populations (Spleen) and thymocytes (Thymus) at 5 weeks of age. This is shown in FIGS. 2a and 2b. In hyper-IgM syndrome due to CD40L deficiency, antigen-independent lymphocyte development is normal. Rather, the hallmark phenotype includes defects in germinal center (GC) formation and antibody responses to T-dependent antigens. Therefore, the ability to form GCs was determined in transgenic and control mice intraperitoneally challenged with sheep red blood cells (SRBC).

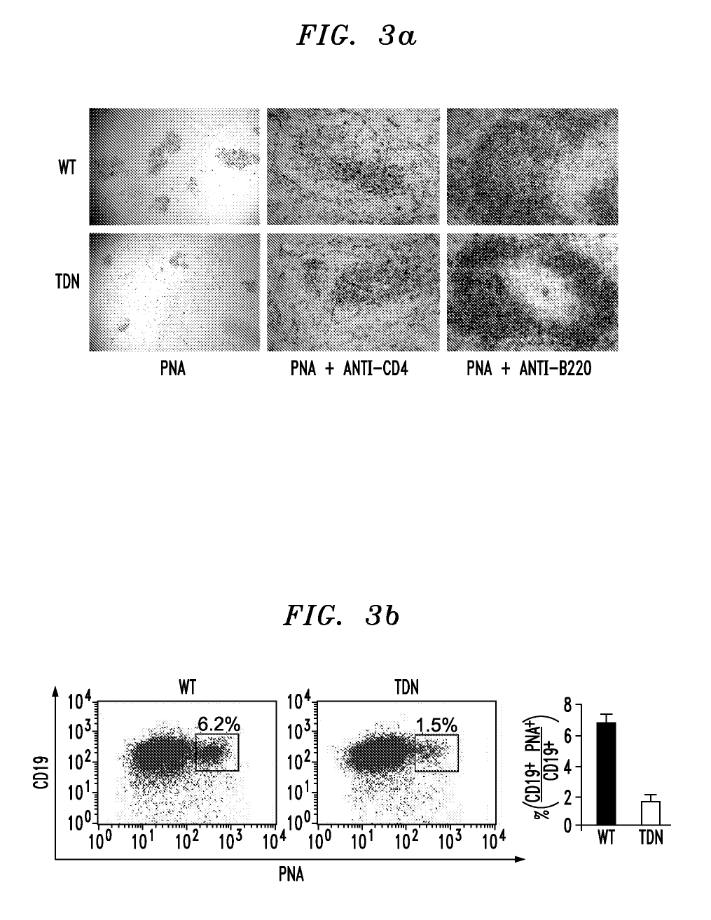

FIGS. 3a and 3b are illustrative graphical views depicting impaired germinal center formation in TDN-transgenic mice immunized with SRBCs, according to an embodiment of the present invention.

Comparison of spleen sections on day 8-9 post inoculation revealed that GC formation was substantially reduced in TDN-transgenic mice compared to controls, with many of the detectable GCs smaller and poorly formed, an example of which is depicted in FIG. 3a (PNA and PNA plus B220 stains; PNA (peanut agglutinin) and B220 (also referred to as B cell antigen of 220 kiloDalton molecular weight) identify GC B cells). This was confirmed in a more quantitative way, for example, by flow cytometry, which showed an approximate 5-fold reduction of CD19-+PNA+ splenic GC B cells compared to controls, as depicted in FIG. 3b. In contrast, FIG. 3a (PNA plus CD4 stains, the latter which identifies CD4 T cells) illustrates that TDN-transgenic T cells are present in the normal, expected locations in the spleens of immunized mice, adjacent to B cells. This is another property of T cells that could have been affected and caused the immune deficiency, but FIGS. 3a and 3b illustrate that it was not such a cause. That is, if T cells cannot make it to the proper place in the spleen, they cannot interact with B cells even if they had expressed CD40L. FIGS. 3a and 3b depict that T cells can make it to the proper place in the spleen, thereby illustrating that this property is normal and therefore does not account for the GC defect.

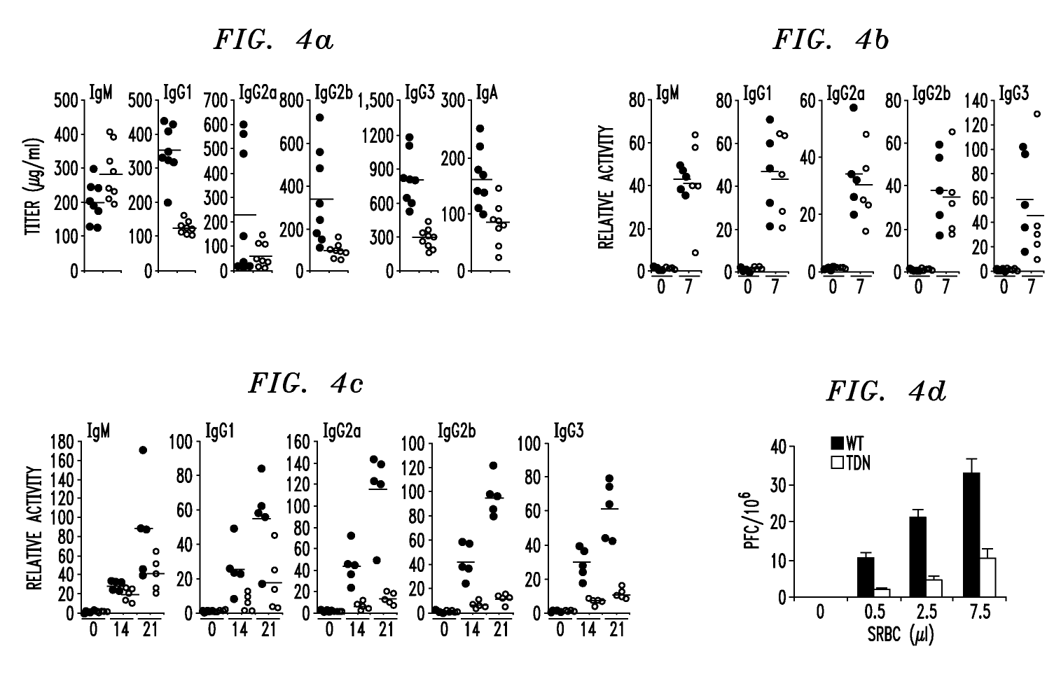

FIGS. 4a through 4d are illustrative graphical views of ELISAs depicting impaired T-dependent, but normal T-independent, humoral (antibody) responses in TDN-transgenic mice, according to an embodiment of the present invention.

Impaired GC formation is indicative of a lapse in T_{H}-cell (helper T cells, primarily CD4+ T cells) dependent B cell activation and is predictive of impaired T-dependent antibody responses. Consistent with this, serum levels of total IgG and IgA isotypes in naive TDN-transgenic mice were reduced compared to controls, whereas total IgM was normal if not slightly elevated, as shown in FIG. 4a. This prediction was then tested directly by evaluating the humoral responses of mice that were challenged with the T-independent and T-dependent antigens TNP-Ficol1 and TNP-KLH, respectively. As shown in FIG. 4b, TNP-specific antibody responses to TNP-Ficol1 were comparable in TDN-transgenic and control mice. However, TDN-transgenic mice failed to mount a characteristic humoral response to the T-dependent antigen TNP-KLH compared to normal, as evidenced by dramatically reduced TNP-specific IgG antibodies at 14 and 21 days post immunization compared to controls, as illustrated in FIG. 4c. Similarly, per capita SRBC-specific plasma cell formation was also proportionally reduced, as shown in FIG. 4d. Thus, TFE3 and TFEB inactivation in T cells mediated by transgenic TDN expression led to poor thymus dependent B cell responses to model antigens in a manner consistent with a CD40L deficiency.

Analysis of TDN-transgenic mice from two other independently generated T cell-specific lines also showed comparable T cell and B cell numbers and distribution but similarly impaired germinal center formation and generation of plasma cells in response to SRBC challenge compared with that of non-transgenic littermates. In these ways, T cell-specific TDN-transgenic mice showed the hallmark phenotypes of hyper-IgM syndrome, in which antigen-independent lymphocyte development and antibody responses to T cell-independent antigens are intact, but germinal center formation and IgG responses to T cell-dependent antigens are defective.

The normal distribution and locations of major T cell populations in unimrnmunized TDN transgenic mice suggests that any other possible target genes of TFE3 and/or TFEB in T cells were not required for T cell development per se beyond the double positive (DP) stage when the TDN protein was first clearly detected (see, for example, FIGS. 2 and 3a). Also unaffected were antibody responses to the thymus independent antigen TNP-Ficoll (FIG. 4), which suggests that any T cell functions that influence this response were largely intact in the absence of TFE3 and TFEB activity.

As detailed below, one or more embodiments of the invention illustrate that defective CD40L expression by TDN-transgenic T cells underlies the humoral immune defect in TDN-transgenic mice.

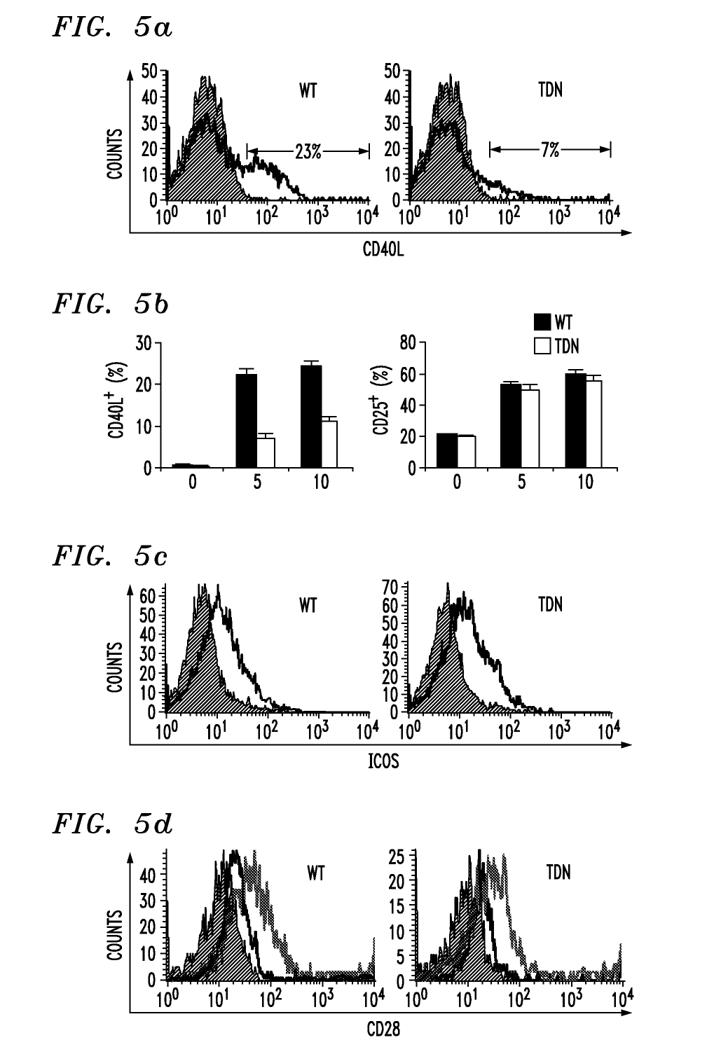

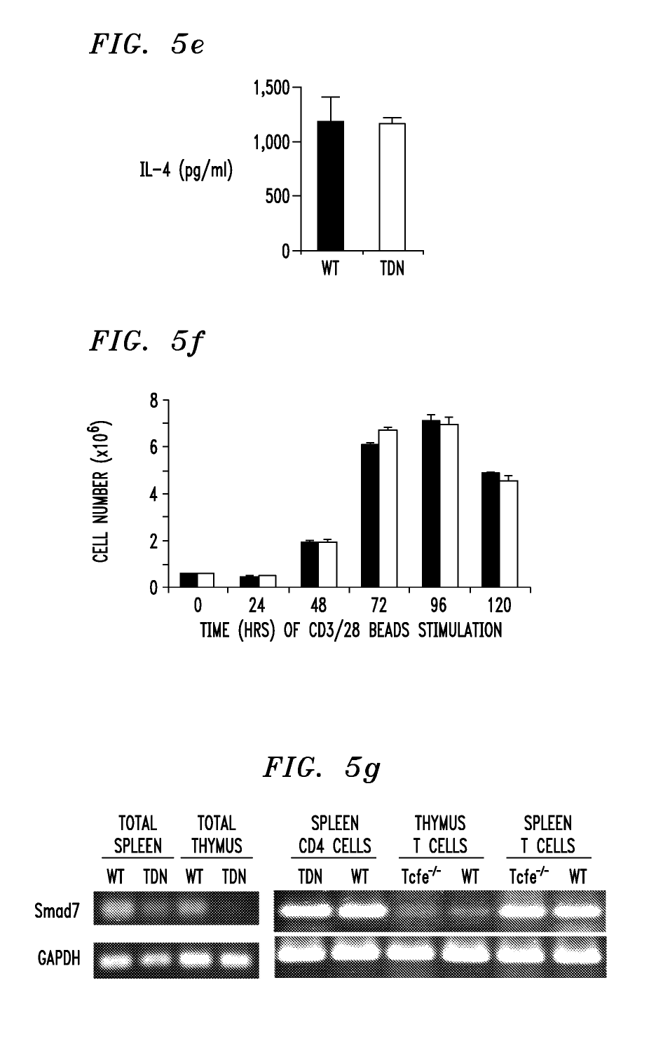

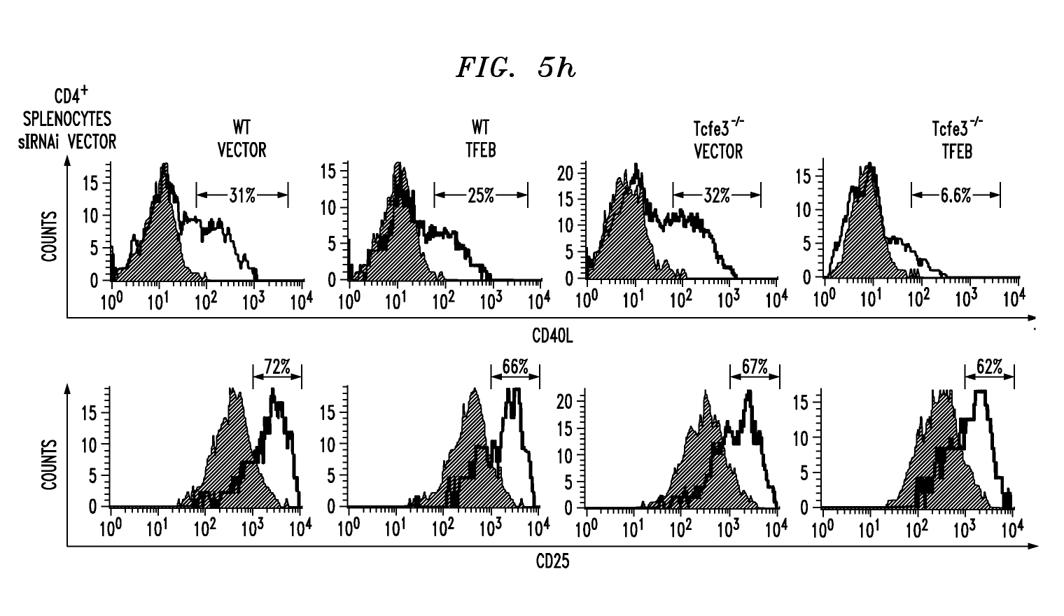

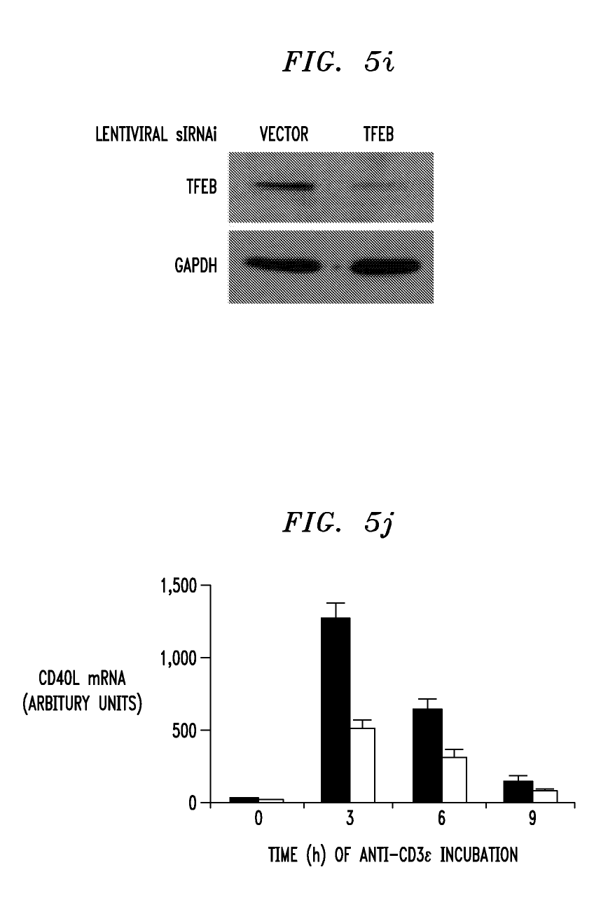

FIG. 5a depicts an illustrative flow cytometry of surface expression of CD40L by control (wild-type (WT)) and TDN-transgenic (TDN) CD4+ splenocytes stimulated, by way of example, with the anti-CD3 mAb and analyzed by flow cytometry for CD40L marker expression, according to an embodiment of the present invention. Filled curves represent CD40L staining of un-stimulated cells, and grey lines represent CD3-stimulated cells. Numbers above bracketed lines indicate CD40L+ cells. Data are representative of more than three experiments.

FIG. 5a depicts how surface expression of CD40L is impaired in TDN-transgenic T cells. The expression of CD40L was much lower on the surface of TDN-transgenic CD4+ T cells than that of non-transgenic (WT) cells after TCR stimulation. Representative data is of T cells from one TDN-Tg line of mice, but is similar in the three lines studied.

However, the induction of other molecules including inducible co-stimulator (ICOS), CD25 CD28, and CD69 was indistinguishable between them, as shown, for example, in the following FIGS. 5b-d (and data not shown).

FIG. 5b depicts an illustrative quantification in bar graph form of histogram data as in FIG. 5a for percent CD4+ T cells positive for CD40L (left) and CD25 (right) after 8 hours of stimulation with various amounts of mAb to CD3 (Anti-CD3), according to an embodiment of the invention. In FIG. 5b, n=6 mice for total CD40L analyses, and n=3 mice for analysis of both CD40L and CD25 in the same experiment. The error bars depicted in the bar graph show the s.e.m. (standard error of the mean) for those values.

FIGS. 5c and 5d depict illustrative flow cytometry histograms of expressions of ICOS and CD28, respectively, showing that their expression is unaffected in TDN-Tg T cells compared to non-Tg (WT), according to an embodiment of the present invention. In FIGS. 5c and 5d, filled curves represent the relative intensity of ICOS or CD28 on freshly isolated CD4+ T cells. Green lines depicted in FIG. 5d represent the intensity on cells left un-stimulated in culture for 2 days. Grey lines depicted in FIG. 5c and grey lines depicted in FIG. 5d represent the intensity on cells stimulated for 2 days with mAb to CD3. Data are representative of three separate experiments.

FIG. 5e depicts an illustrative graphical representation of ELISA measurements of IL-4 secretion, according to an embodiment of the present invention.

As shown in FIG. 5e, in vitro IL-4 secretion by TDN-transgenic T cells is normal. CD4+ T cells from control and transgenic mice were cultured with plate-bound anti-CD3 mAb for 2 days, followed by IL-2 for 7 days, and then re-stimulated with anti-CD3 without IL-2 for 2 days. Data for n=3 independent experiments, and standard error bars are shown.

FIG. 5f depicts an illustrative bar graph showing that the proliferative response of TDN-Tg CD4 T cells in vitro to mitogenic stimulation by CD3/CD28 beads is indistinguishable from control non-Tg CD4 T cells. Purified T cells were placed in culture, CD3/CD28 beads added, and cell counts taken over a time course. Data for n=3 independent experiments, and standard error bars are shown. We show that T cell proliferation in response to CD3 and CD29 co-stimulation is indistinguishable between non-Tg and TDN-Tg T cells. These proliferation data support one of the major the assertions depicted in FIGS. 2, 3a and 5, namely, that TDN-Tg CD4 T cells (that is, T cells that lack TFE3/TFEB function) are relatively normal.

FIG. 5g depicts an illustrative RT-PCR showing that Smad7 expression is unaffected in TDN-Tg and Tcfe3−/− splenic CD4 T cells. Smad7 is analyzed here because it is regulated by TFE3 and TFEB in non-hematopoietic cells.

In sum, FIG. 5 of the present invention teaches us that inactivation of TFE3 and TFEB had no effect on TCR-dependent CD25, CD69, or CD28 expression, IL-4 secretion, IL-2 responsiveness or CD3/CD28 responsiveness of CD4+ T cells, or Smad7 expression. The data therefore suggest that the effect of TFE3 and/or TFEB inactivation was relatively restricted to CD40L and did not globally affect T cell responses to stimulation, in contrast to deficiencies in some NFAT and NFkB subunits.

One or more embodiments of the present invention illustrate that CD40L expression by CD4 T cells depends on endogenous TFE3 or TFEB. That is, they are mutually redundant activators of CD40L expression in CD4 T cells.

FIG. 5h depicts illustrative flow cytometry histograms showing CD40L and CD25 staining profiles of un-stimulated (filled curve) and TCR-stimulated (line overlay) green fluorescent protein (GFP+) cells from normal and Tcfe3−/− mice (for three independent experiments), in which GFP shows cells that express either a control virus or a virus that expresses an inhibitory (interfering) stem-loop RNA that blocks TFEB synthesis, according to an embodiment of the present invention. Primary CD4+ splenic T cells were isolated from normal and Tcfe3−/− mice infected with the indicated stem-loop-interfering RNA/GFP lentivirus and incubated with IL-2 for about 4-6 days. Cells were then stimulated with plate-bound anti-CD3 mAb for about 8 hours and analyzed for CD40L or CD25 surface expression by flow cytometry.

Germline Tcfe3−/− mice have no reported defects in immune function, and the very early embryonic lethality caused by germline TFEB-deficiency has precluded the study of Tcfeb−/− T cells. Therefore, the remaining models consistent with the biochemical, promoter, and genetic data prior to the experiment in FIG. 5h were that TFEB itself is important for CD40L expression, or there exists a functional redundancy between TFE3 and TFEB with respect to CD40L expression. However, the TDN, by blocking both TFE3 and TFEB, does not allow discrimination between these models. To address this issue, in FIG. 5h CD40L expression was evaluated in primary splenic CD4+ T cells from wild-type (WT) and Tcfe3−/− mice rendered TFEB-deficient by repressing endogenous TFEB expression via retroviral introduction of a TFEB-specific stem-loop interfering RNA (slRNA).

FIG. 5i depicts an illustrative representation of an immunoblot confirming down-regulation of TFEB in CD4+ T cells infected with a retrovirus that expresses the inhibitory stem-loop TFEB RNA as in FIG. 5h, according to an embodiment of the present invention. FIG. 5i depicts an immunoblot of extracts from sorted GFP+ cells infected with a control (left) or TFEB-slRNA lentivirus, for TFEB and GAPDH. The immunoblot is representative of two independent experiments.

With continued reference to FIG. 5h, induction of CD40L by TCR-stimulation with an anti-CD3 mAb was comparable between wt T cells infected with the control or TFEB slRNA lentiviruses and Tcfe3−/− T cells infected with the control lentivirus, suggesting TFEB itself was not critical if TFE3 was present. In contrast, CD40L induction on Tcfe3−/− T cells infected with the TFEB slRNA vector was significantly impaired compared to control infected and wt cells. Induction of CD25 was unaffected by the TFEB slRNA in all cases. In addition, growth of wt and Tcfe3−/− T cells expressing the TFEB slRNA was indistinguishable from control-infected cells. These results support the model that TFE3 and TFEB have important and mutually redundant roles in the TCR-dependent induction of CD40L by primary mouse splenic T cells. Data presented herein excludes the possibility that impaired CD40L expression was a secondary consequence of MiT-deficient T cell development or persistent TDN protein expression.

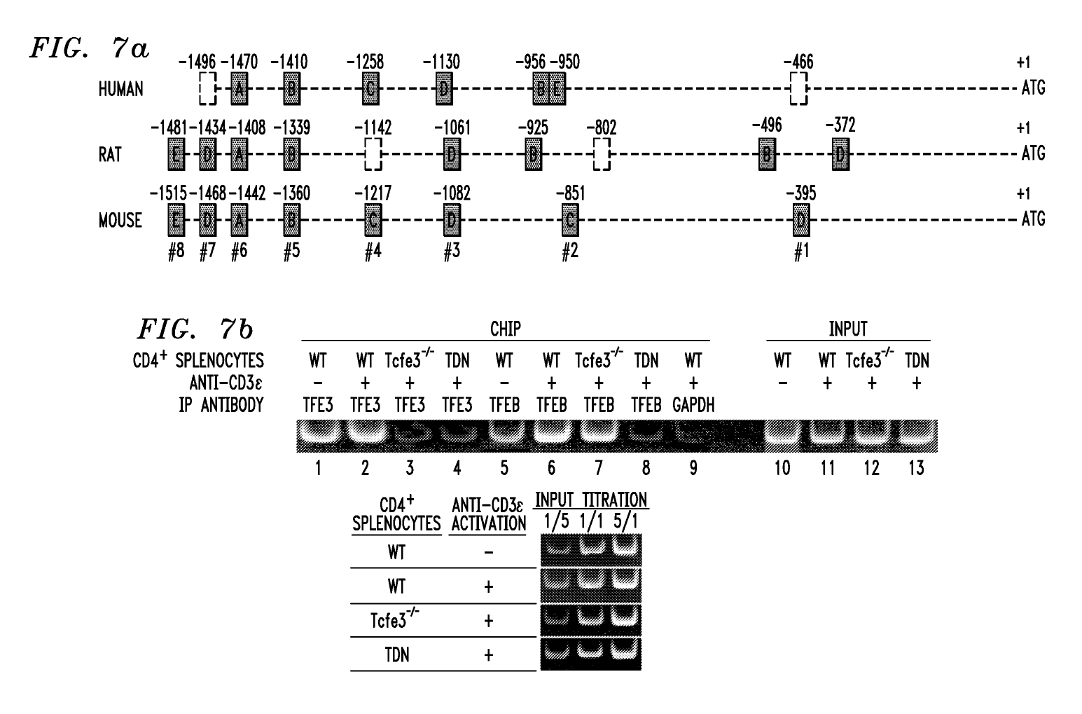

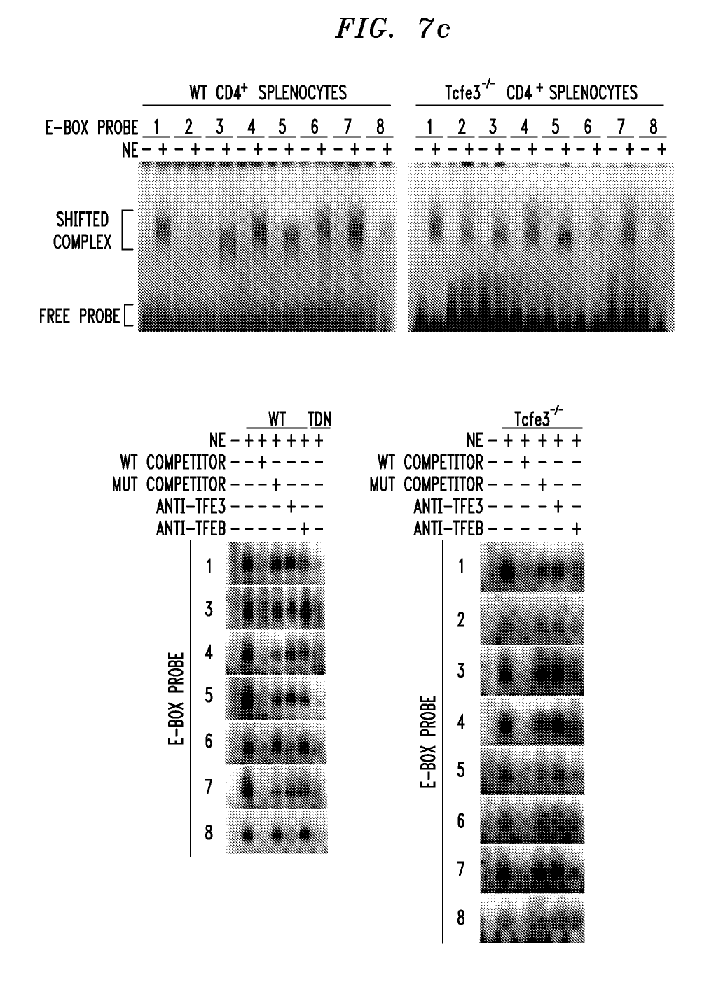

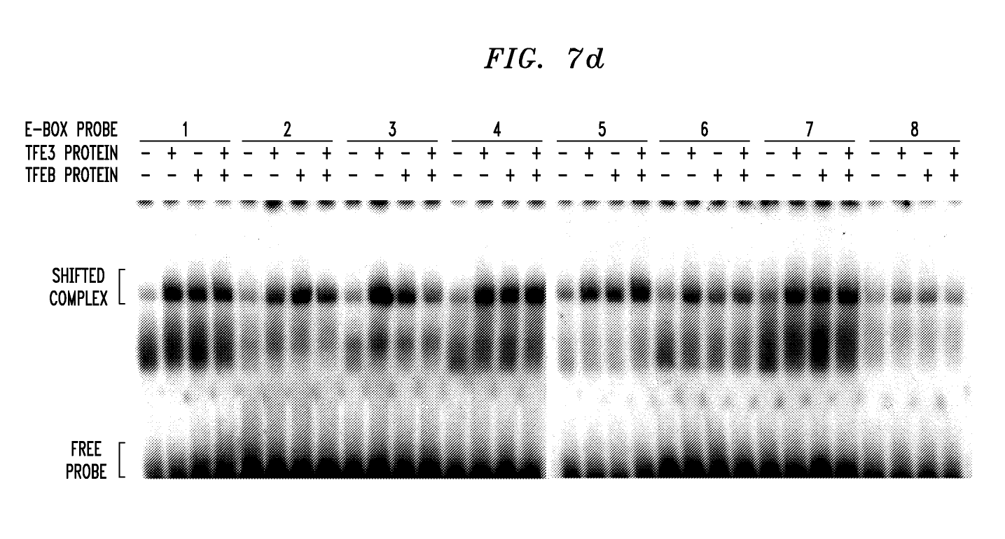

These one or more embodiments of the present invention therefore represent the first known demonstration of a physiological functional redundancy between TFE3 and TFEB and the first known demonstration of their role in controlling T cell function via their joint regulation of CD40L (see, for example, FIGS. 5, 7, 8, and 9). It remains possible that under certain conditions each has a unique contribution to the activation of CD40L based on observations that only TFEB was induced after TCR stimulation of primary cells (FIG. 1a and 1b) and that TFEB could bind independently from TFE3 to the CD40L promoter (FIG. 7).

The present invention teaches us that TFE3 and TFEB control the expression of CD40L by directly controlling transcription of the gene encoding CD40L (Cd40lg and CD40LG).

FIG. 5j depicts an illustrative real-time RT-PCR analysis of RNA isolated from CD4+ T cells from wild-type or TDN-transgenic littermate mice at various times after incubation with mAb to CD3 (horizontal axis), according to an embodiment of the invention.

In FIG. 5j, the illustrative bar graph shows the relative amounts of mRNA encoding CD40lg from CD4+ T cells from wild type (wt) (black bars) or TDN-transgenic littermate mice (white bars) at the indicated times after incubation with the anti-CD3 mAb. By way of example only, and without loss of generality, real-time RT-PCR analysis of CD40lg RNA isolated from TDN-transgenic and control CD4+ splenic T cells at various times after TCR-stimulation showed a reduction of Cd40lg transcripts in TDN-Tg T cells at the induction peak to one-third that of non-Tg (control) cells, with unchanged overall kinetics (FIG. 5j). These data substantiate the model that TFEB and TFE3 directly activate CD40lg at the transcriptional level in vivo. Data from n=3 independent experiments and standard error bars are shown. Similar results were obtained with T cells from other independently generated, T cell-specific, TDN-transgenic lines.

In sum, one or more embodiments of the present invention teach a fundamental relationship between the transcription factors TFE3 and TFEB and T cell function via their direct regulation of CD40L expression. The data are most consistent with a mutually redundant role of these molecules with respect to controlling CD40L transcription, as a combined TFE3/TFEB deficiency inhibited TCR-dependent CD40L induction in the primary T cell culture systems, whereas individual deficiencies had either a minor or no measurable effect (see, for example, FIG. 5h). A degree of functional redundancy has also been observed with NFAT components, as a combined deficiency of NFATc1 and NFATc2 resulted in the greatest impairment in CD40L expression compared to individual deficiencies. The transgenic approach to inactivate endogenous TFE3 and TFEB via T cell specific expression of the TDN protein was therefore a key and advantageous strategy to address the redundancy and also served to circumvent the embryonic lethality of germline TFEB deficiency by directing expression to somatic cells.

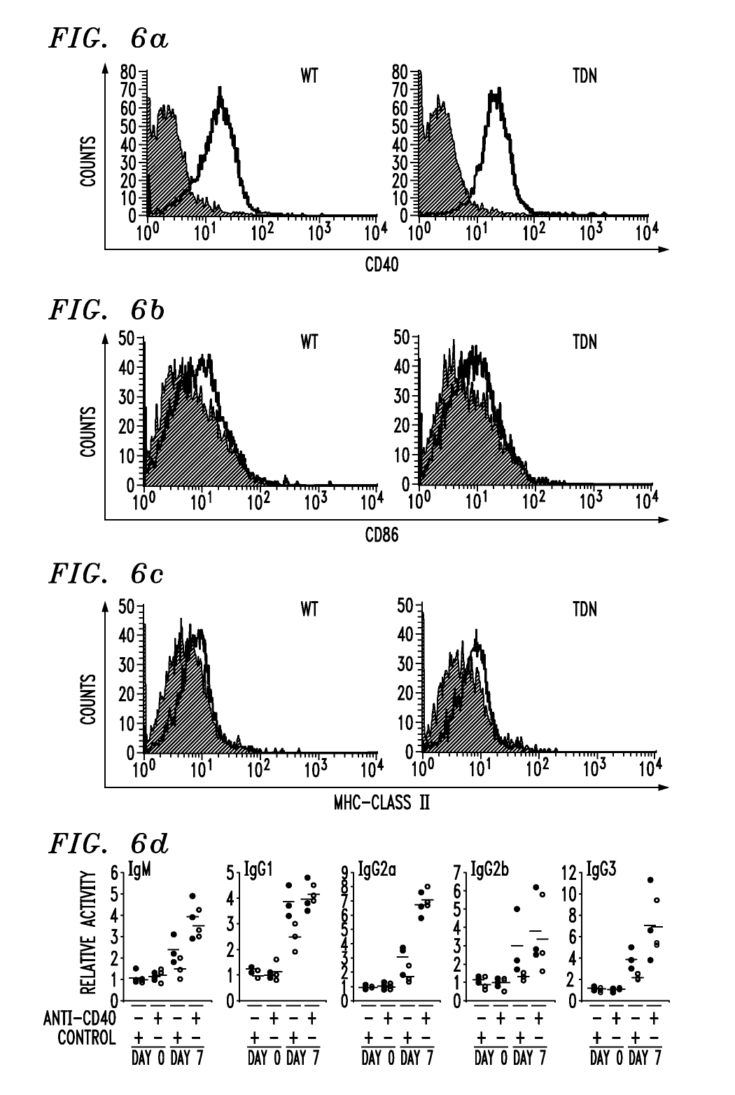

One or more embodiments of the present invention teaches us that B cells from the T-cell specific TDN-Tg mice (FIGS. 1e-1h) are not themselves defective and are capable of responding normally to antigen and CD40 signaling (FIG. 6). Consistent with that, CD19+ B cells from TDN-transgenic mice and non-transgenic mice had similar expression of CD40 and responded similarly to in vitro stimulation, including the CD40-dependent induction of CD86 and major histo-compatibility complex (MHC) class II expression. Moreover, an agonist monoclonal antibody (mAb) to CD40, administered during immunization with TNP-KLH, enhanced day-7 TNP-specific IgG titers of both non-transgenic and TDN-transgenic mice, but most notably rendered the IgG responses indistinguishable from each other. In contrast, IgG antibody titers in mice treated with the isotype-matched control mAb were lower, and TDN-transgenic mice also had lower IgG responses than those of non-transgenic mice. These data indicate that B cell responses to T cell help and other aspects of T cell help critical for this T cell-dependent antibody response were intact in the TDN-transgenic mice and supported the conclusion that the humoral immune defect in TDN-transgenic mice was T cell intrinsic and was due to defective CD40L expression.

FIG. 6a depicts an illustrative flow cytometry histogram showing equivalent CD40 expression in B cells from non-transgenic and TDN-transgenic mice, according to an embodiment of the present invention. Filled curves represent unstained cells, and grey lines represent cells stained with mAb to CD40.

FIGS. 6b and 6c epict illustrative flow cytometry histograms showing that expressions of CD86 and MHC class II respectively, by B cells from TDN-Tg and non-Tg mice are equivalent, after stimulation of B cells with mAb to CD40, according to an embodiment of the present invention. In FIGS. 6b and 6c, filled curves represent CD86 or MHC class II staining on un-stimulated cells, and grey lines on stimulated cells.

FIG. 6d depicts illustrative ELISA analyses measuring IgG responses to TNP-KLH in non-transgenic and TDN-transgenic mice treated with an agonist mAb to CD40 during immunization, according to an embodiment of the present invention. TNP-KLH is an experimental antigen to which an antibody response requires T-cell derived CD40L to B cells. Control mice (filled circles) and TDN-transgenic mice (open circles) were immunized intraperitoneally once with 100 micrograms of TNP-KLH plus 100 micrograms of either isotype-matched control antibody (control) or mAb to CD40 (3/23), delivered intravenously. The same amount of each mAb was administered once a day over 7 days. Data represent TNP-specific IgG isotypes from serum samples obtained on days 0 and 7.

One or more embodiments of the present invention illustrate that TFE3 and TFEB bind to and activate the CD40L promoter.

FIG. 7a depicts an illustrative schematic alignment of CD40L promoters from humans, mice and rats, showing where TFE3 and TFEB bind to and activate the CD40L promoter, according to an embodiment of the present invention.

The human, mouse, and rat CD40L promoters contain a number of E-box like sequences, a subset of which matches known optimal μE3-binding sites for MiT proteins and which are present in the CD40L promoter regions from each species. By way of example only, FIG. 7a depicts an illustrative schematic alignment of CD40L promoters from humans, mice and rats, showing where TFE3 and TFEB bind to and activate the CD40L promoter.

E-boxes are categorized as either ones that match known and optimal MiT transcription factor binding sites and that are present in all promoters (gray boxes with letters) or as non-conserved and non-optimal (dotted open boxes). The optimal MiT sites are further subcategorized based on the precise sequence of the site, as represented by letters A through E. Nucleotide positions of boxes are numbered, with A of the ATG translation start site equal to +1, as will be understood by those skilled in the art.

FIG. 7b upper panel depicts an illustrative Chromatin immunoprecipitation (ChIP) of a Cd40lg promoter fragment by anti-TFE3 and anti-TFEB antibodies, according to an embodiment of the present invention. The lower panel depicts illustrative controls for ChIP assays in the upper panel of FIG. 7b, according to an embodiment of the present invention. Titration of input DNA confirms amplification conditions in the ChIP assay are in the semi-quantitative, linear range. Input material ( 1/900th of total) from the upper panel of FIG. 7b was serially diluted as indicated above each lane and amplified in parallel with the experimental samples from FIG. 7b.

Chromatin immunoprecipitation (ChIP) assays were used to determine whether endogenous TFE3 and TFEB could bind to the CD40L promoter in vivo. ChIP analysis of un-stimulated and TCR-stimulated primary CD4+ splenic T cells from wild-type (wt) mice showed that TFE3 and TFEB each bound to a fragment of the murine Cd40lg promoter under both resting and stimulated conditions. FIG. 7b depicts exemplary Chromatin immunoprecipitation (ChIP) of a Cd40lg promoter fragment by anti-TFE3 and anti-TFEB antibodies. By way of example only, primary CD4+ splenocytes from normal and Tcfe3−/− mice were un-stimulated or stimulated with anti-CD3, as indicated above each lane (for example, lanes 1-13) in FIG. 7b, upper panel. Shown is a semi-quantitative PCR analysis of starting material (Input, lanes 10-13) and immunoprecipitated material (ChIP, lanes 1-9) from the indicated sources and with the indicated Abs. CHIP using an anti-GAPDH antibody (lane 9) served as an additional negative control for background band intensity. The exemplary data shown in FIG. 7b is representative of three independent experiments.

Material detected in the anti-TFE3 ChIP sample from Tcfe3−/− cells defined background band intensity and was equivalent to the irrelevant antibody ChIP control (for example, compare lanes 3 and 9 in FIG. 7b, upper panel). Interestingly, whereas TFE3 binding did not change, TFEB binding appeared to increase after T cell activation by TCR cross-linking in a manner that mirrored in the increase of TFEB protein after TCR stimulation (see, for example, lanes 5 and 6 in FIG. 7b; FIG. 1a). The ability of TFEB to bind the fragment could be independent of TFE3, as TFEB recruitment to the Cd40lg promoter was unaffected in Tcfe3−/− cells (for example, lane 7 in FIG. 7b, upper panel). Neither TFE3 nor TFEB binding to the Cd40lg promoter was detected by ChIP in TDN-transgenic CD4+ splenic T cells, thus documenting an effective inactivation of endogenous TFE3 and TFEB DNA binding activity in those T cells by the transgenically expressed TDN protein (see, for example, FIG. 7b upper panel, lanes 4 and 8). FIG. 7c (described below) also illustrates how TDN expression in T cells in vivo via transgenesis blocks TFE3 and TFEB binding to the CD40L promoter using a different methodology.

FIG. 7c depicts illustrative electrophoretic mobility-shift assays (EMSAs) to evaluate the binding of native TFE3 and TFEB proteins to the individual MiT sites in the Cd40lg promoter, according to an embodiment of the present invention. Nuclear extracts (NE) from stimulated wild-type and Tcfe3−/− splenic CD4+ T cells were incubated with radio-labeled oligonucleotides spanning a single MiT E-box (sites 1-8) and complexes were resolved by native gel electrophoresis. The top two panels show relative E-box-binding activity in extracts made from WT (left) or Tcfe3−/− (right) CD4 T cells. The markers on the left point out shifted complexes and free probe. The bottom EMSAs represent cold oligonucleotide competition and antibody-interference assays to determine the identity and specificity of E-box-binding complexes in cell extracts.

Each of the panels in the lower sets of EMSAs shows binding activity to one particular E-box probe from WT (left, first six lanes), TDN-Tg (left, last lane) or Tcfe3−/− (right panels) CD4 T cells. Excess unlabeled E-box oligonucleotides, with wild-type (WT) or with point mutations (Mut), and anti-TFE3 or anti-TFEB were included in some binding reactions (+; above lanes). The exposures for the bottom panels were optimized for each probe. Data are representative of at least three independent experiments.

FIG. 7d depicts illustrative electrophoretic mobility shift assays (EMSAs) of nuclear extracts of human embryonic kidney (HEK293) cells transfected with control (−), TFE3, or TFEB pEBB (pEBB is a plasmid used by many labs to express genes in mammalian cells; the “B” in EBB stands for El alpha, the name of the mammalian gene from where the promoter was obtained) expression vectors showing TFE3 and TFEB binding to the MiT sites from the Cd40lg promoter, according to an embodiment of the present invention. As shown in FIG. 7c, EMSAs with nuclear extracts from primary CD4+ T cells established that native TFE3 and TFEB bound to the multiple MiT consensus E-boxes in the Cd40lg promoter, although to differing relative degrees and sometimes with preferential binding of one or both subunits to individual sites. Similar results were obtained here (FIG. 7d) by analyzing DNA-binding activities in extracts of HEK293 cells over-expressing TFE3 or TFEB. We demonstrated the specificity of the protein-DNA interactions by including TFE3- or TEEB-specific antibodies that interfered with DNA binding.

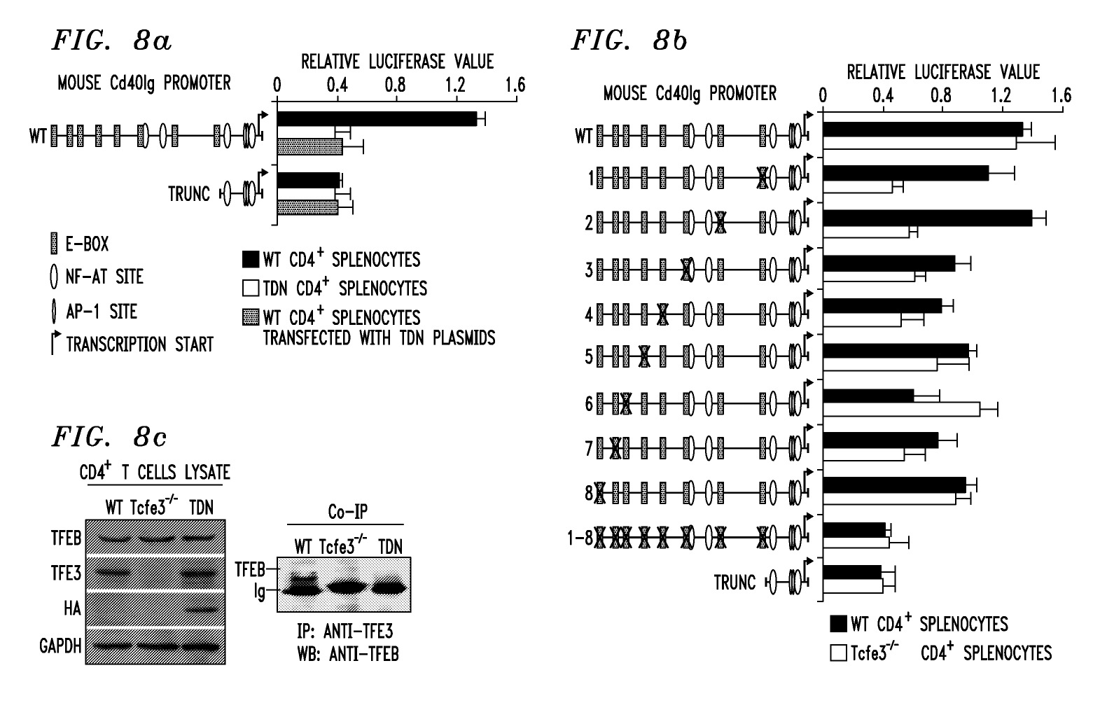

FIG. 8a depicts a bar graph plotting the relative activities of Cd40lg promoter fragments containing (full-length) or lacking (truncated) the MiT E-boxes and in the presence or absence of the TDN protein, as measured by (firefly) luciferase reporter gene assays, and shows that Cd40lg promoter activity depends on endogenous TFE3, TFEB and s MiT boxes to enhance promoter activity, according to an embodiment of the present invention. Primary CD4+ splenic T cells from wild-type and TDN-transgenic mice were transfected with mouse Cd40lg promoter-luciferase constructs, either full-length (from the ATG to −1535 base pairs upstream) or truncated (to −382) lacking the MiT sites (rectangles). A separate plasmid that contains Renilla luciferase, molecularly distinct from firefly luciferase and whose expression was dependent on a promoter that does not require TFE3 and TFEB, was co-transfected to provide an internal transfection control for normalizing firefly luciferase values. Then, 2 days later, cells were stimulated for 8 hours with mAb to CD3, then were collected and lysed for luminometry. Data represent relative luciferase values (normalized to Renilla values). Standard error bars are shown, and in FIG. 8a, n=three independent transfections using cells prepared from three separate sets of wild-type and TDN-transgenic mice.

FIG. 8b depicts illustrative contributions of individual and combinations of MiT E-box sites to promoter activity, according to an embodiment of the present invention. Point mutations abrogating binding of TFE3 and/or TFEB binding (‘X’) were introduced into E-box sites (numbered 1-8; left margin) in the context of the full-length mouse Cd40lg promoter. Primary splenic CD4+ T cells from wild-type and Tcfe3−/− mice were transfected, stimulated and analyzed for luciferase activity as in described in FIG. 8a.

Point mutations that abrogated the binding of TFE3 and/or TFEB to individual sites also attenuated Cd40lg promoter activity in primary wild-type and TFE3-deficient CD4+ T cells (FIG. 8b), although to differing degrees. A full-length construct containing mutations of all eight sites had the greatest effect on promoter activity, reducing it to the activity of the truncated promoter. Individual mutation of sites three through seven had a less severe but measurably attenuating effects in both wild-type and TFE3-deficient T cells. In contrast, mutation of site one or two had no effect in wild-type cells but had a substantial effect in TFE3-deficient T cells. That result was consistent with the preferential binding of TFEB from wild-type extracts to site one and the greater relative binding of TFEB to site two than to all other sites in the absence of TFE3.

The differential importance of some sites in TFE3-deficiency versus wild-type cells may indicate that the TFE3 and TFEB redundancy was not complete or that sites had differential responsiveness to TFE3 or TFEB protein abundance. These results indicated that all sites in some context could act in concert for full TFE3- and/or TFEB-dependent enhancement of Cd40lg promoter activity. Thus, we infer that TFE3 or TFEB binding is critical for achieving the physiological regulation of CD40L expression necessary for T cell-dependent antibody responses.

FIG. 8c depicts that native TFE3 and TFEB can form heterodimers and that the TDN protein blocks this interaction, (i.e., that heterodimerization, by way of example, is an interaction that is blocked by the TDN protein), according to an embodiment of the present invention. In FIG. 8c, the graphical depiction on the left represents an immunoblot of stimulated CD4+ splenic T cell extracts from WT, Tcfe3−/− and TDN-Tg mice for TFE3, TFEB, TDN (HA), and GAPDH protein expression. In FIG. 8c, the graphical depiction on the right represents extracts from the same sources that were incubated with anti-TFE3, immune complexes that were precipitated, and immuno-precipitates resolved on SDS-PAGE and blotted with anti-TFEB. In wild-type CD4+ T cells, native TFE3 and TFEB were immuno-precipitated together (FIG. 8c lane 1), suggesting that, in addition to TFE3 homodimers and TFEB homodimers, that TFE3/TFEB heterodimers can form and may represent a distinct DNA binding species. That interaction was blocked in TDN-transgenic T cells (FIG. 8c lane 3). These data confirmed functional inactivation of endogenous TFE3 and TFEB by the transgenically expressed TDN.

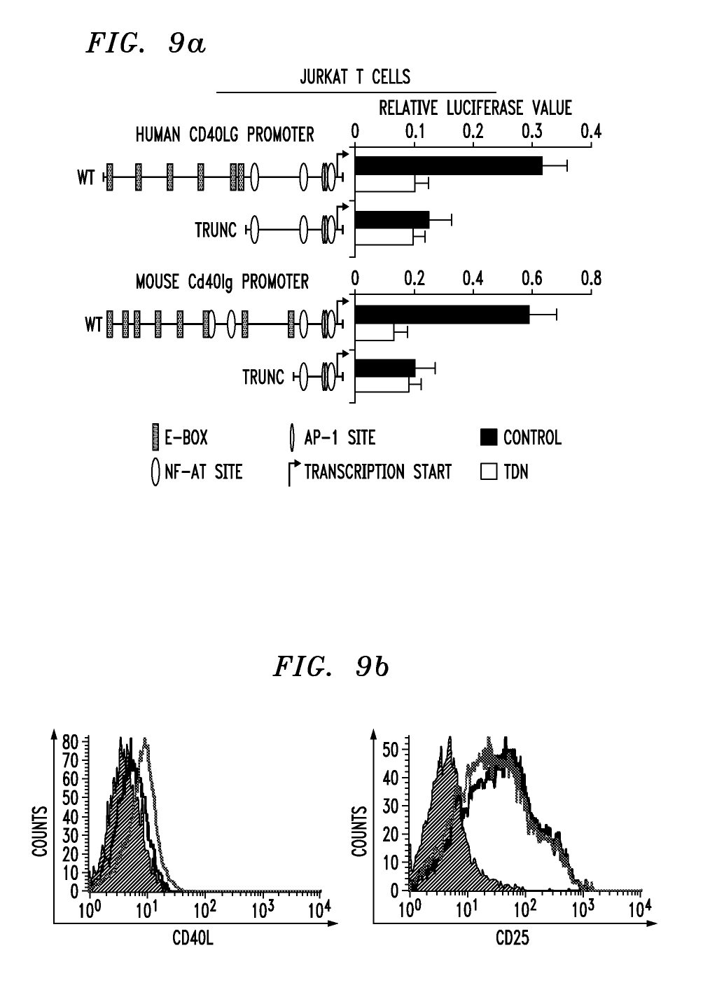

FIG. 9a is a bar graph depicting relative levels of Cd40lg and CD40LG promoter activity in human T cells (Jurkat) as represented by exemplary normalized luciferase values for each condition and promoter construct with respect to control renilla values for n=3 and standard error bars, according to an embodiment of the present invention. By way of example only, maximal Cd40lg and CD40LG promoter activity required an upstream fragment that contains conserved MiT boxes and endogenous TFE3 and/or TFEB.

To determine whether endogenous TFE3 and TFEB were important for CD40L promoter activity in human T cells, the firefly luciferase reporter gene assay was used. In this assay, murine Cd40lg and human CD40LG promoter fragments were linked to the luciferase gene and transiently transfected into the human T cell line Jurkat. Jurkat T cells were transfected either with full-length (−1.5 kB upstream: human −1562, mouse to −1535 relative to the ATG) or truncated (−382, mouse; −944, human) CD40L promoter-luciferase constructs with control or TDN-expressing plasmid and with Renilla as an internal control. About 2-4 hours later, cells were mock treated or treated with a combination of phorbol myristate acetate (PMA) and ionomycin (lono), namely, PMA/iono, as above, then harvested 20 hours later for luciferase expression.

Any contribution of endogenous TFE3 and TFEB in Jurkat T cells to CD40L promoter activity was evaluated by comparing promoter activity in the presence or absence of the trans-dominant-negative (TDN) inhibitory protein, which can simultaneously inactivate both endogenous TFE3 and TFEB proteins. As mentioned previously, the TDN protein contains the TFE3 HLH/Zip (helix-loop-helix/leucine zipper) dimerization domains but lacks the DNA binding BR and transcription activation domains and thus forms heterodimers with MiT proteins that are incapable of binding DNA (FIG. 8c).

With continued reference to FIG. 9a, transient transfection of the reporters into Jurkat T cells showed that the activity of the full-length mouse Cd40lg and human CD40LG promoters exhibited about 3-fold higher activity at the induction peak following PMA/iono stimulation than the corresponding truncated promoters that lacked the consensus MiT binding sites. However, simultaneous expression of the TDN protein reduced the activity of the full-length promoters to the levels of the truncated promoter fragments, whereas the activities of the truncated promoters were unaffected by TDN expression. Thus, deletion of an upstream promoter fragment of the Cd40lg and CD40LG genes that contained the predicted MiT consensus sites rendered the promoters unresponsive to inhibition of endogenous TFE3 and TFEB by the TDN.

The truncated promoters were still responsive to stimulation, which was attributed to the proximal NFAT/AP-1 site as has been reported (see, for example, Lobo, F. M., Xu, S., Lee, C. & Fuleihan, R. L, “Transcriptional activity of the distal CD40 ligand promoter,” Biochem Biophys Res Commun, pp. 245-250, 279 (2000), Lindgren, H., Axcrona, K. & Leanderson, T., “Regulation of transcriptional activity of the murine CD40 ligand promoter in response to signals through TCR and the co-stimulatory molecules CD28 and CD2,” J Immunol 166, pp. 4578-4585 (2001), and Parra, E., Mustelin, T., Dohlsten, M. & Mercola, D., “Identification of a CD28 response element in the CD40 ligand promoter,” J lmmunol 166, pp. 2437-2443 (2001), the disclosures of which are incorporated by reference herein). Therefore, the TDN did not block the activity of these other transcription factors, and established that endogenous TFE3 and/or TFEB were important for maximal induction of CD40LG and Cd40lg promoter activity in human T cells via MiT site-containing sequences.

FIG. 9b is an illustrative graphical view depicting how inhibition of native TFE3 and TFEB activity by expression of trans-dominant negative protein (TDN) blocks endogenous CD40L induction after activation of Jurkat T cells, according to an embodiment of the present invention.

In accordance with an embodiment of the invention, FIG. 9b illustrates that inhibition of TFE3 and TFEB activity by expression of the TDN via the pEBB plasmid blocks CD40L induction after activation of human Jurkat T cells. By way of example only, T cells were transiently transfected with the indicated pEBB-GFP-expression construct (control or TDN-expressing) and two days later stimulated with PMA/iono. About 12 hours later, cells were analyzed, for example, by flow cytometry, or alternative analysis methods, for surface expression of CD40L (left histogram) and CD25 (right histogram). In both histograms, shown are exemplary CD40L and CD25 expression profiles of GFP+ (plasmid transfected) cells that were un-stimulated (filled profiles), cells that received the control plasmid and were stimulated (red line-overlays), and cells that received the TDN plasmid (green line overlay). Data shown is representative of at least three independent experiments.

TDN protein expression selectively inhibited the induction of endogenous CD40L by Jurkat T cells dependent on stimulation by phorbol myristate acetate (PMA) plus ionomycin (iono), whereas the induction of CD25 was unaffected (FIG. 9b). Therefore, one or more embodiments of the present invention teaches us that endogenous TFE3 and/or TFEB are important for maximum induction of Cd40lg and CD40LG promoter activity and endogenous gene expression in mouse and human T cells and that they have evolutionarily conserved functions in that capacity (FIGS. 9a and 9b). In other words, what was defined in mouse CD4 T cells is also operational in human T cells (even though they are transformed).

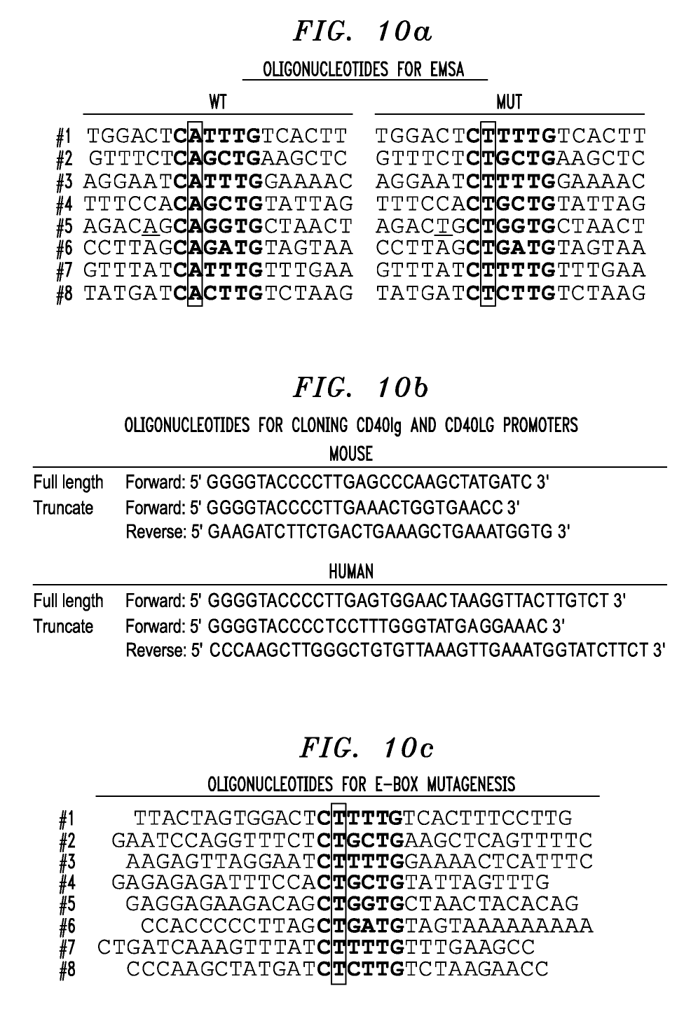

FIG. 10a depicts a table illustrating oligonucleotides for EMSA in FIG. 7, according to an embodiment of the present invention. The oligonucleotides are very short sequences of DNA that are the same MiT DNA sequences as they exist in the Cd40lg promoter. This is why and how we used them to show that TFE3 and TFEB proteins interact with the MiT sites in the Cd40lg promoter. FIG. 10a is merely an exemplary embodiment, and one or more embodiments of the present invention are not limited to the MiT sites used therein. One or more embodiments of the present invention any implement other MiT sites to bind to TFE3 and TFEB that do not come from the Cd40lg promoter.

FIG. 10b depicts an exemplary table illustrating oligonucleotides for cloning Cd40lg and CD40LG promoters, according to an embodiment of the present invention.

FIG. 10c depicts an exemplary table illustrating oligonucleotides for E-box mutagenesis for the analyses in FIG. 8b, according to an embodiment of the present invention.

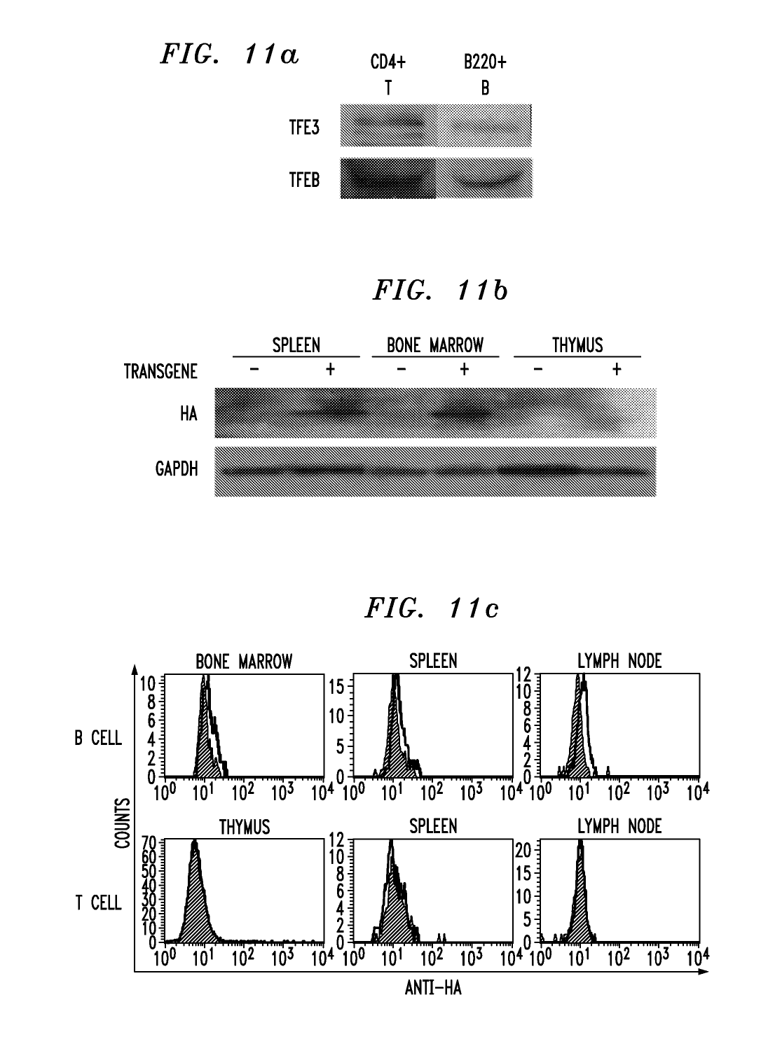

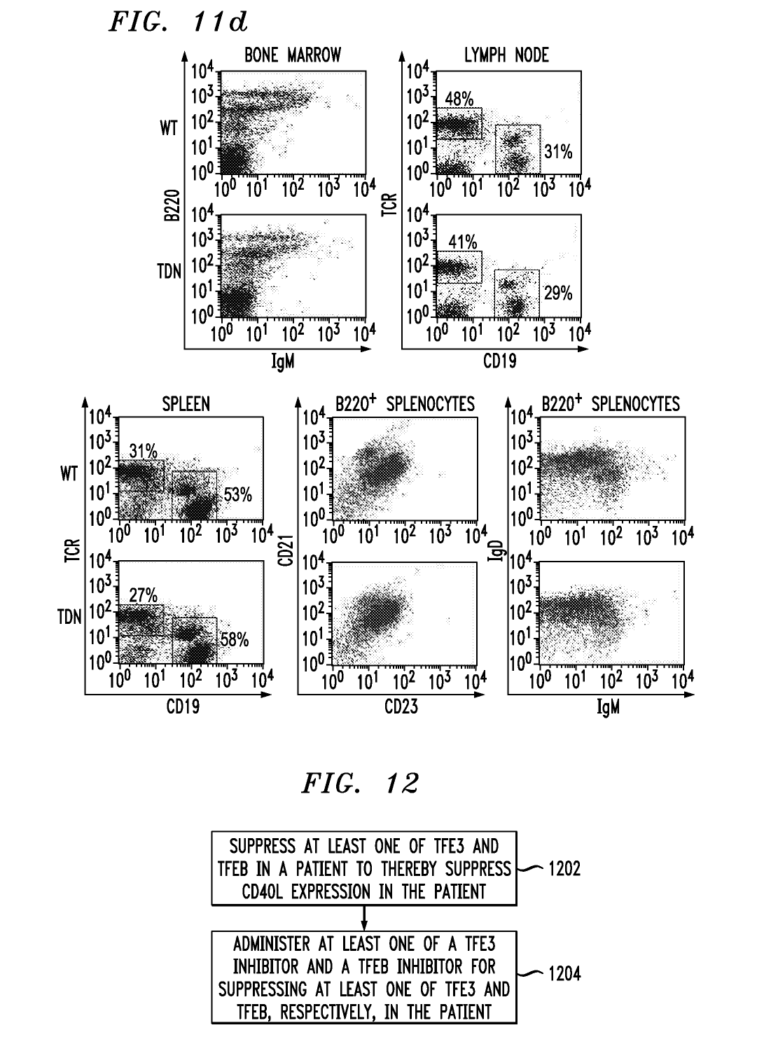

FIGS. 11a-11c show that TDN expression in the B cell lineage does not block B cell development.

Furthermore, one or more embodiments of the invention include experiments that are from different lines of transgenic mice in which the TDN inhibitor is expressed in B cells, which make antibodies, and T cells in these mice are normal and do not express the TDN inhibitor. These lines are called “B-cell-specific TDN-Tg mice.”

FIG. 11a depicts a representative immunoblot showing that TFE3 and TFEB proteins are expressed in B cells, according to an embodiment of the present invention.

FIG. 11b depicts representative immunoblots probing for TDN expression in lymphoid tissues from a different line of EμPμ transgenic mice that are shown here to express the TDN in B cells but not T cells (so called “B cell-specific TDN-Tg mice”), according to an embodiment of the present invention.

FIG. 11c depicts representative flow cytometry, also showing that these mice express the TDN protein only in B, but not T cells, according to an embodiment of the present invention.

FIG. 11d depicts representative flow cytometry analyses showing that major B and T cell populations in these “B cell-specific TDN-Tg” mice are normal, according to an embodiment of the present invention.

Lines of EμPμ-TDN transgenic mice were identified that expressed the TDN exclusively in B cells, not T cells. The immunoblot and flow cytometry shown in FIG. 11b and 11c is proof of this principle showing that the TDN inhibitor is made only in B cells in these lines, and that TFE3 and TFEB proteins are expressed in B cells. The flow cytometry analysis of lymphocytes in these mice shows that, remarkably, B cell development is normal (FIG. 11c). This is important because it suggests that systemic administration of a hypothetical TFE3 and/or TFEB inhibitor will not effect the production of B cells.

FIG. 12 is a flow chart illustrating a technique for treating at least one of an immunological disease and an inflammatory disease in a patient, according to an embodiment of the present invention. In a preferred embodiment of the present invention, the technique for treating an immune disease in a patient includes step 1202, suppressing at least one of TFE3 and TFEB in a patient to thereby suppress CD40L expression in the patient.

Many human diseases are associated with abnormal CD40L expression (see, for example, Danese, S. & Fiocchi, C., “Platelet activation and the CD40/CD40 ligand pathway; mechanisms and implications for human disease,” Crit Rev Immunol 25, pp. 103-121 (2005), and Schattner, E. J., “CD40 ligand in CLL pathogenesis and therapy,” Leuk Lymphoma 37, pp. 461-472 (2000), which are incorporated by reference herein). For example, CD40L expression is typically elevated on T cells from SLE and RA patients. Inappropriate or undesirable expression of CD40L by T cells (or B cells and possibly other cell types) can cause autoantibody production by B cells and/or directly or indirectly (for example, via autoantibody complexes) trigger inflammatory responses mediated by the cellular immune system that destroy tissues and organs. CD40L is also necessary for B cells to become transformed by the Epstein Barr Virus. Interfering with CD40L-CD40 interactions—either genetically (in mice) or pharmacologically (for example, via CsA or interfering CD40L mAb) has had proven efficacy in mouse models and some efficacy in the clinic for ameliorating symptoms of and thus treating many autoimmune/inflammatory diseases.

One or more embodiments of the present invention teaches that TFE3 and TFEB control the expression of CD40L in CD4 T cells and thus provides a new way to control abnormal or undesirable CD40L expression in disease states. In addition, TFE3 and TFEB can each be regulated by the MAPK pathway, which can be activated by stimulation of multiple receptors including the TCR and the IL-15 receptor, and IL-15 is elevated in RA. Thus, it is possible that the activity of TFE3 and TFEB can be regulated by MAPK activation with respect to CD40L expression by these and other receptors under normal and disease states. Moreover, given the broad distribution of TFE3 and TFEB expression in different cell types, CD40L expression in other cell types, including, but not limited to, B cells, monocytes and platelets, may similarly depend on TFE3 and TFEB and the effects of disease state on their expression in these cells. For example, B cell abnormalities in some autoimmune diseases may be due to homotypic stimulation via co-expression of CD40 and CD40L on abnormal B cells.

In one or more embodiments of the present invention, the step of suppressing at least one of TFE3 and TFEB may include selectively inactivating TFE3 and/or TFEB, respectively.

Also, in one or more embodiments of the present invention, the step of suppressing at least one of TFE3 and TFEB may alternatively include the step of blocking an ability of at least one of TFE3 and TFEB to interact with DNA.

Furthermore, in one or more embodiments of the present invention, the step of suppressing at least one of TFE3 and TFEB includes the step of blocking an ability of at least one of TFE3 and TFEB synthesis. Suppressing TFE3 and/or TFEB may be achieved, in accordance with an aspect of the invention, by blocking TFE3 and/or TFEB synthesis, respectively, via an interfering RNA (which includes sIRNA, as, by way of example only, in an embodiment of the invention). Blocking TFE3 and/or TFEB synthesis would leave other MiT proteins untouched and would be predicted to have the least invasive, least collateral off-target effects.

In one or more embodiments of the present invention, the step of suppressing at least one of TFE3 and TFEB includes at least one of blocking an interaction between TFE3 and TFEB, blocking TFE3 and TFEB from forming dimers, and blocking TFE3 and TFEB from binding with DNA. Blocking TFE3 and TFEB from forming dimers may comprise blocking TFE3 and TFEB from forming dimers with each other, blocking TFE3 from forming dimers with itself, and/or blocking TFEB from forming dimers with itself.

Also, in accordance with one or more embodiments of the present invention, the step of suppressing at least one of TFE3 and TFEB in the patient to thereby suppress CD40L expression in the patient may include the use of the suppressing TDN protein. Blocking TFE3 and/or TFEB protein activity via a TDN-like molecule or oligonucleotide MiT binding site DNA binding blockers may also affect Mitf and TFEC. However, in one or more embodiments of the present invention, molecular inhibitors of this type may have great efficacy if used locally, for example, at the site of a transplant or in bead form and released locally. As described herein, TDN can experimentally block Mitf and TFEC as illustrated, for example, by the depiction herein that Mitf is expressed in thymocytes and un-stimulated T cells. Additionally, Mitf and TFEC may be capable of binding to the MiT sites in the CD40L gene promoters in other cell types that express these molecules.

In one or more embodiments of the present invention the step of suppressing at least one of TFE3 and TFEB in the patient to thereby suppress CD40L expression in the patient may include some type of interfering RNA (this includes the interfering stem-loop RNA, as was used here, and double stranded oligonucleotide RNAi).

In one or more embodiments of the present invention, the step of suppressing at least one of TFE3 and TFEB in the patient to thereby suppress CD40L expression in the patient may include some type of agent that would block the activation of TFE3 and/or TFEB by MAPK phosphorylation or prevent interactions between TFE3 and TFEB and upstream or downstream effectors that depend on this phosphorylation.

In one or more embodiments of the present invention, the technique for treating at least one of an immunological disease and an inflammatory disease in a patient may comprise gene therapy, which may also benefit by including TFE3 and TFEB binding sites in the vector to promote appropriate expression of genes within the gene therapy vector in the patient.

In one or more embodiments of the present invention, the immune disease includes at least one of an autoimmune disease and an inflammatory disease.

In one or more embodiments of the present invention, the technique for treating an immune disease in a patient may also include step 1204, administering at least one of a TFE3 inhibitor and a TFEB inhibitor for suppressing at least one of TFE3 and TFEB, respectively, in the patient.

In one or more embodiments of the present invention, at least one of the TFE3 inhibitor and the TFEB inhibitor is administered systemically. Also, in one or more embodiments of the present invention, at least one of the TFE3 inhibitor and the TFEB inhibitor is administered as an inhalant.

Furthermore, in one or more embodiments of the present invention, at least one of the TFE3 inhibitor and the TFEB inhibitor is administered subcutaneously. As way of example and not limitation, the TFE3 inhibitor and the TFEB inhibitor may be administered at site of inflammation and/or rejection.

Materials and Methods

Cell Culture, CD4 T Cell Purification and T Cell Activation

By way of example only, CD4+ T cells were purified from spleens of 5 week old mice with a mouse CD4 negative isolation kit (Dynal Biotech) to greater than or equal to about 96% purity as determined by flow cytometry. T cells were cultured in RPMI supplemented with 10% (volume/volume) heat-inactivated FCS (fetal calf serum) (Gibco), and 50 μM β-mercaptoethanol (Sigma). For the lentiviral stem loop RNAi experiment, 20 units/ml mouse IL-2 (Roche) was added to the culture medium.

For TCR stimulation, the purified mouse spleen CD4+ T cells—either cultured in IL-2 for 4-5 days (slRNAi) or freshly isolated—were incubated in 5-10 μg/ml anti-CD3 mAb (anti-CD3ε mAb 145-2C11, BD Pharmingen)-fixed plates for ˜8 hr before harvesting for detection of surface CD40L and CD25 expression.

For CD28 expression, the cells were incubated with or without 10 μg/ml anti-CD3 for 48 hr after initiation of the culture. For ICOS expression analysis, T cells were incubated for 48 hours with 10 μg/ml anti-CD3 following the manufacturer's instructions (BD Pharmingen). For the real-time PCR, freshly isolated and purified spleen CD4+ T cells were incubated in 10 μg/ml anti-CD3 fixed plates in RPMI/FBS/BME for 0 hour (hr), 3 hr, 6 hr, and 9 hr before harvesting for RNA extraction and reverse transcription. Approximately 2×10⁷ cells were used per time point.

For stimulation of Jurkat cells, PMA (20 ng/ml final) and ionomycin (1.5 μM final) were added to the RPMI/FBS/BME culture medium for 12 or 24 hours before harvesting for fluorescent antibody staining and reporter gene assays. IL-4 cytokine analysis was done as described (24). Briefly, CD4+ T cells were removed from anti-CD3 stimulation after 48 hr, cultured for 7 days in Roswell Park Memorial Institute medium (RPMI)/Fetal bovine serum (FBS)/beta-mercaptoethanol (BME) containing 100 units/ml mouse IL-2 (Roche), then re-stimulated at 10⁵ cells/well in 96-well plate bound with 5 μg/ml anti-CD3 without IL-2 for 48 hours. Supernatants were collected for quantitative enzyme-linked immunosorbent assay (ELISA).

For B cell purification and B cell stimulation, splenic B cells were purified with a mouse B cell Negative Isolation kit (Dynal Biotech) such that 94% or more of the cells were CD19+. Purified B cells were then analyzed directly for CD40 expression or were stimulated for 48 hours with mAb to CD40 (3/23; BD Pharmingen) and then were analyzed for CD86 and MHC class II surface expression by flow cytometry.

Generation and Identification of Transgenic Mice

To create the TDN-transgenic mice, the 3′ hemagglutin (HA) epitope-tagged TDN CDNA was subcloned into an irnmunoglobutin heavy chain gene enhancer and promoter based transgene cassette (EμPμ) that can direct expression in T cells, B cells or both, depending on the line. Fragments that contained the transgene cassette were excised from the plasmid backbone, purified and microinjected into fertilized FVB oocytes as described in the SUNY-Downstate Transgenic Facility (see, e.g., Hogan, B., Beddington, R., Costantini, F. & Lacy, E, “Manipulating the Mouse Embryo: A Laboratory Manual,” Cold Spring Harbor Laboratory Press, Cold Spring Harbor, N.Y., 1994, which is incorporated by reference herein).

Founder mice were identified by Southern blotting and PCR of genomic DNA from tails. Transgenic mice analyzed were the progeny of transgenic animals that had been back-crossed at least four generations onto the C57BL/6 strain. Five independent lines were derived that were confirmed to express TDN transgene only in T cells. Three lines were evaluated for recapitulation of impaired induction of CD40L in primary CD4+ T cells. All showed the same defect, and results from one line is shown. All transgenic mice used were 5-6 weeks old and hemizygous for the transgene, and non-transgenic control mice were sex-matched littermates of transgenic mice. The mice were housed under SPF (specific pathogen free) conditions according to guidelines approved by the Division of Laboratory Animal Resources.

Lentivirus-Delivered Stem-Loop RNAi

The control and TFEB sIRNA lentiviruses are described in Huan et al. and viral particles produced according to published protocols (see, for example, Rubinson, D. A. et al., “A lentivirus-based system to functionally silence genes in primary mammalian cells, stem cells and transgenic mice by RNA interference,” Nat Genet 33, pp. 401-406 (2003), which is incorporated by reference herein). Briefly, the pLentiLox 3.7 plasmid inserted with TFEB MRNA targeting stem-loop RNAi was co-transfected into 293 cells with the packaging vectors pMDLg/pRRE (“p”, refers to “plasmid,” and the MDLg and RRE are conventional names for viral genes that encode a function necessary to build viral particles, so called “helper” functions), CMV-VSVG (CVM refers to transcription activation sequences from the cytomegalo virus that are built into plasmids to express other genes, in this case the G protein from VSV; and VSVG is a name for viral genes that encode a function necessary to build viral particles) and RSV-Rev (RSV refers to transcription activation sequences from the Rous sarcoma viruses that are built into plasmids to express other genes, in this case the Rev protein from HIV; and Rev is a name for viral genes that encode a finction necessary to build viral particles). The viruses in culture media were concentrated by centrifugation, and used for infecting purified mouse spleen CD4+ T cells. The infected T cells were cultured with 20 units/ml IL-2 for 4-5 days before evaluating infection efficiency and anti-CD3 stimulation.

Flow Cytometry

To detect cell surface markers, single cell suspensions were treated with Fc receptor block (2-4G2, BD Pharmingen), and were stained with anti-CD19, B220, TCRβ, CD40L, CD25, CD28, CD4, CD8, ICOS and/or CD86 conjugated to FITC, PE, or Cy-Chrome (BD Pharmingen) or with fluorescein isothiocyanate-conjugated anti-MHC class II (Caltag). For detection of the TDN protein in transgenic lymphocytes, cells were treated with Fix & Perm Kit (Caltag), before being stained with FITC conjugated anti-HA mAb (3F10, Roche). Cells were analyzed on a FACScan (A BD company name for a particular model of flow cytometer: Fluorescence Activated Cell Sorter/Scan) with CellQuest™ software (a trademark of Becton Dickenson).

Chromatin Immunoprecipitation Assays (ChIP)

The ChIP assay was done as described (see, for example, Huan, C., Sashital, D., Hailemariam, T., Kelly, M. L. & Roman, C. A. J., “Renal carcinoma associated transcription factors TFE3 and TFEB are leukemia-inhibitory factor-responsive transcription activators of E-cadherin,” J Biol Chem 13, p. 13 (2005)), with the following modifications: Purified 1.5×10⁷ mouse spleen CD4+ T cells with or without anti-CD3 stimulation for 8 hr were used for each immunoprecipitation, with 5 μg of the following antibodies: anti-TFE3 mAb (G138-312, BD Pharmingen), anti-TFEB (ab2636, abcam), and anti-GAPDH (MAB374, Chemicon). Then, 10% of the precipitated DNA and 0.11% of the input DNA were used as templates for each PCR using the following conditions: 1 minute (min) at 94° C., 1 min at 61° C., and 1.5 min at 72° C. for 29 cycles. The PCR products were separated by 5% acrylamide gel electrophoresis. The primers used for amplifying mouse CD40 ligand promoter region are: forward, 5 CACAGACAGCATCCCTAGCA 3, and reverse 5 CTAAGCTGAG GCCAAACCAC 3.

Real-Time Reverse Transcriptase Mediated (RT)-PCR

Total RNA from purified CD4+splenic T cells was isolated with TRI reagent (TR-118, MAR) and contaminant DNA was removed by DNase I (AMP-D1, Sigma) according to the manufacturer s instructions. Total RNA (5 μg) was reverse-transcribed and PCR amplified with QuantiTect SYBR Green RT-PCR kit (Qiagen) in an Opticon Continuous Fluorescence Detector (MJ Research). Detection of mouse CD40L and GAPDH mRNAs were determined by real-time RT-PCR using following primers, for CD40L, 5′ AAAATGGG AAACAGCTGACG 3′, and 5′ GGTATTTGCCGCCTTGAGTA 3′, for GAPDH, 5′ TCACCACCATGGAGAAGGC 3′, and 5′ GCTAAGCAGTTGGTGGTGCA 3′. CD40L mRNA expression was normalized to GAPDH mRNA.

Enzyme-Linked Immunosorbent Assay (ELISA) and Immunizations

Total serum levels of IgM, IgG1, IgG2a, IgG2b, IgG3 and IgA were determined using mouse ELISA quantitation kits (Bethyl Lab). Bound Ig was detected using a tetramethylbenzidine (TMB) peroxidase substrate system (KPL). The T cell-independent type II and T cell-dependent immunizations and immune responses assays were done as described (see, for example, Yamazaki, T. et al., “Essential immunoregulatory role for BCAP in B cell development and function,” J Exp Med 195, pp. 535-545 (2002), which is incorporated by reference herein).

In vivo stimulation of B cells with mAb to CD40 was done as described (see, for example, McAdam, A. J. et al. “ICOS is critical for CD40-mediated antibody class switching,” Nature 409, 102-105 (2001), which is incorporated by reference herein) with modifications. Mice were immunized intraperitoneally with 100 μg TNP-KLH (Biosearch Technologies) in complete Freund's adjuvant (Difco) and were injected intravenously each day for seven days after immunization with 100 μg anti-CD40 (mAb 3/23; BD Pharmingen) or the isotype control rat IgG2a κ (mAb R35-95; BD Pharmingen), and then blood was collected to obtain serum. Serum Igs specific for TNP (Trinitrophenol) were measured in Immulux HB plates (DYNEX) coated with TNP-BSA (Trinitrophenol-bovine serum albumin conjugate) (Biosearch Technologies) with ELISA quantitation kits (Bethyl Lab).

ELISA was performed with EL405 auto plate washer (BIO-TEK), the plates were read at 450 nm with μQuant microplate spectrophotometer (BIO-TEK), and data was analyzed with KCjunior microplate data analysis software (BIO-TEK). For IL-4 cytokine analysis, T cells culture supernatants were assayed for cytokine content by Mouse IL-4 Immunoassay Elisa kit (R&D) following manufacturer's protocol.

Immunocytochemical Analysis of Germinal Centers

Mice were immunized intraperitoneally with 7.5 μl SRBC (Colorado Serum) in 200 μl PBS. The spleens were harvested 8 days after immunization and fixed in 10% formalin. Germinal centers were stained as previously described (see, for example, Cattoretti, G. et al., “BCL-6 protein is expressed in germinal-center B cells,” Blood 86, pp. 45-53 (1995), which is incorporated by reference herein) with modification.

Briefly, antigen-retrieved slides were blocked with 3% pig serum and avidin/biotin blocking kit (Vector Lab), incubated with 1 μg/ml of biotin-conjugated PNA (Sigma) for more than 2 hr, and then incubated with biotin-conjugated goat anti-PNA (Vector Lab) for 45 min. Subsequently, the sections were treated with 0.1% NaN₃ and 0.3% H₂O₂ for 30 min to block the endogenous peroxidase before staining with 1:400 diluted HRP-conjugated avidin (Dako) for 20 min. After washing, the sections were incubated with HRP developing solution for 20 min, and then embedded with glycerol gelatin. All staining procedures were performed at room temperature.

Electrophoretic Mobility-Shift Assay Analysis

These assays were done as described below. Nuclear extracts from anti-CD3-activated CD4+ T cells or transfected HEK293 cells (5 μg) were incubated on ice for 30 minutes with a ³²P-labeled oligonucleotide spanning an E-box site from the mouse Cd40lg promoter in 25 mM HEPEs, pH 7.9, 50 mM KCl, 4% (weight/volume) Ficoll, 5 μM ZnCl₂, 0.1 mM dithiothreitol, 0.02% (volume/volume) Nonidet-P40, 5 mM MgCl₂, 10 μg/ml of BSA and 10 ng/μl of poly(dI:dC) (deoxyinosine:deoxycytidine). Nonspecific binding was assessed in the presence of a 100-fold excess of unlabeled oligonucleotide corresponding to the labeled oligonucleotide with or without E-box mutation and 2.5 μg anti-TFE3 (BD Pharmingen) or anti-TFEB (Santa Cruz). Samples with antibody were incubated for an additional 30 minutes. Reaction products were separated by native 5% (weight/volume) PAGE at 4° C. and were visualized by autoradiography.

CD40L Promoter Reporter Gene Plasmid Construction, Transient Transfection, and Reporter Gene Assay

The 5 extended (“full-length”: to −1535 bp and −1562 bp, relative to the mouse and human ATG, respectively) and truncated mouse and human CD40L promoter fragments (to −382 bp and −944 bp from the ATG, respectively) were PCR amplified from genomic DNA and inserted into the pGL3 luciferase reporter vector (Promega). Mouse Cd40lg promoter reporter gene plasmids with E-box mutations (CANNTG to CTNNTG) were constructed by site-directed mutagenesis (Stratagene) with specific primers. Jurkat cells were transfected by electroporation with 10 μg of CD40L promoter reporter gene plasmid, 0.05 μg renilla control plasmid and 8 μg TDN expressing plasmid for each sample, and subsequently activated with PMA and ionomycin as described (see, for example, Lobo, F. M., Xu, S., Lee, C. & Fuleihan, R. L., “Transcriptional activity of the distal CD40 ligand promoter,” Biochem Biophys Res Commun 279, pp. 245-250 (2000), incorporated herein by reference).

Firefly luciferase (Luc) and Renilla reniformis luciferase (Rlluc) activities were measured from cell extracts with the Dual Luciferase Reporter Assay System (Promega) and TD-20/20 Luminometer (Turner Designs). Primary mouse CD4 T cells were transfected with 2.5 μg of Cd40lg promoter reporter gene plasmids, 0.01 μg of RLIuc and 1.5 μg of empty pEBB or pEBB-TDN expression plasmid for each sample using the mouse T cell Nuclefector kit (Amaxa). Then, two days after transfection, the cells were activated for eight hours with 5 μg/ml of mAb to CD3ε and then were lysed for luminometry as described herein. Luciferase activity was always normalized to Rlluc activity. In all experiments, the total amount of pEBB expression vector DNA was equalized by balancing cDNA-containing pEBB with empty pEBB.

Protein Detection

Purified mouse CD4+ splenic T cells and Jurkat T cells after or without activation were lysed in detergent buffer (150 mM NaCl, 20 mM Tris pH7.4, 1% Triton-100, 0.1% SDS), supplemented with Complete Protease Inhibitor Tablets (Roche). Protein was transferred to PVDF membrane (Immobilon P). The following antibodies were used for western: anti-mouse TFE3 (BD-Pharmingen), anti-TFE3 (Santa Cruz Biotechnology), anti-TFEB (abeam), anti-GAPDH (CHEMICON), rabbit anti-goat IgG peroxidase conjugate (Sigma), and goat anti-mouse IgG peroxidase conjugate (Sigma).

The blots were developed by enhanced chemiluminescence (ECL). Coimmunoprecipitation was done according to a protocol from BD-Pharmingen. Purified CD4+ splenocytes (2×107) were lysed in immunoprecipitation buffer (1% (weight/volume) Triton X-100, 150 mM NaCl, 10 mM Tris, pH 7.4, 1 mM EDTA, 1 mM EGTA, pH 8.0, 0.2 mM sodium orthovanadate, 0.5% (weight/volume) Igepal and protease inhibitor ‘cocktail’ (Roche)). Cell lysates were pre-cleared with protein G-agarose beads (Roche) and then were incubated overnight with 5 μg mAb to TFE3 (BD-Pharmingen). Immunoprecipitates were collected on protein G-agarose beads and were washed before elution in sample buffer for protein detection as described herein.

Plaque Forming Cells

The spleen IgM anti-SRBC response was assayed 5 days after intraperitoneal administration of 0.5-7.5 μl SRBC in 200 μl PBS following the protocol of the Jerne plaque assay (see, for example, Jerne, N.K. et al., “Plaque forming cells: methodology and theory,” Transplant Rev 18, pp. 130-191 (1974), incorporated by reference herein).

While the present invention has been described in accordance with the treatment of immune diseases and disorders described herein, it is to be understood that the teachings of the present invention are generally applicable to any diseases or disorders necessitating the regulation of CD40L expression. Thus, the teachings of the present invention should not be construed as being limited to the treatment of any particular disease or disorder.

Although illustrative embodiments of the present invention have been described herein, it is to be understood that the invention is not limited to those precise embodiments, and that various other changes and modifications may be made by one skilled in the art without departing from the scope or spirit of the invention.

What is claimed is:

1. A method of treating at least one of an immunological disease and an inflammatory disease in a patient, the method comprising the step of suppressing at least one of TFE3 and TFEB in the patient to thereby suppress CD40L expression in the patient.

2. The method of claim 1, wherein the step of suppressing at least one of TFE3 and TFEB comprises the step of selectively inactivating at least one of TFE3 and TFEB, respectively.

3. The method of claim 1, wherein the step of suppressing at least one of TFE3 and TFEB comprises the step of blocking an ability of at least one of TFE3 and TFEB to interact with DNA.

4. The method of claim 1, wherein the step of suppressing at least one of TFE3 and TFEB comprises the step of blocking an ability of at least one of TFE3 and TFEB synthesis.

5. The method of claim 1, wherein the step of suppressing at least one of TFE3 and TFEB comprises the step of blocking an interaction between TFE3 and TFEB.

6. The method of claim 1, wherein the step of suppressing at least one of TFE3 and TFEB comprises the step of blocking TFE3 and TFEB from forming dimers.

7. The method of claim 6, wherein the step of blocking TFE3 and TFEB from forming dimers comprises blocking TFE3 and TFEB from forming dimers with each other.

8. The method of claim 6, wherein the step of blocking TFE3 and TFEB from forming dimers comprises blocking TFE3 from forming dimers with itself and blocking TFEB from forming dimers with itself, respectively.

9. The method of claim 1, wherein the step of suppressing at least one of TFE3 and TFEB comprises the step of blocking TFE3 and TFEB from binding with DNA.

10. The method of claim 1, wherein the step of suppressing at least one of TFE3 and TFEB in the patient to thereby suppress CD40L expression in the patient comprises inactivating at least one of TFE3 and TFEB via TDN protein.

11. The method of claim 1, wherein the step of suppressing at least one of TPFE3 and TFEB in the patient to thereby suppress CD40L expression in the patient comprises blocking at least one of TFE3 and TFEB synthesis via an interfering RNA.

12. The method of claim 1, further comprising the step of administering at least one of a TFE3 inhibitor and a TFEB inhibitor for suppressing at least one of TFE3 and TFEB, respectively, in the patient.

13. The method of claim 12, wherein at least one of the TFE3 inhibitor and the TFEB inhibitor is administered systemically.

14. The method of claim 12, wherein at least one of the TFE3 inhibitor and the TFEB inhibitor is administered as an inhalant.

15. The method of claim 12, wherein at least one of the TFE3 inhibitor and the TFEB inhibitor is administered subcutaneously.

16. The method of claim 15, wherein the TFE3 inhibitor and the TFEB inhibitor is administered at a site of at least one of inflammation and rejection.

17. The method of claim 1, wherein the immunological disease comprises an autoimmune disease.

18. The method of claim 1, wherein the step of suppressing at least one of TFE3 and TFEB comprises the step of blocking a transcriptional activity of at least one of TFE3 and TFEB, respectively.

19. The method of claim 18, wherein the step of blocking a transcriptional activity of at least one of TFE3 and TFEB comprises inhibiting phosphorylation of TFE3 and TFEB.

20. The method of claim 18, wherein the step of blocking a transcriptional activity of at least one of TFE3 and TFEB comprises preventing an interaction with at least one other molecule which controls transcription.
